(19) United States
(12) Patent Application Publication
(10) Pub. No.: US 2023/0146923 A1
(43) Pub. Date: May 11, 2023

(54) COMPOSITIONS AND METHODS FOR INHIBITION AND TARGETING OF P97

(71) Applicant: California Institute of Technology, Pasadena, CA (US)

(72) Inventors: Tsui-Fen Chou, Pasadena, CA (US); Shan Li, Pasadena, CA (US); Feng Wang, Pasadena, CA (US); Nadia Houerbi, Pasadena, CA (US)

(21) Appl. No.: 17/982,901

(22) Filed: Nov. 8, 2022

Related U.S. Application Data
(60) Provisional application No. 63/276,905, filed on Nov. 8, 2021.

(51) Int. Cl. C12Q 1/6886; C12N 15/113; C07K 16/40; C12N 9/22; A61P 35/00

(52) U.S. Cl. CPC C12Q 1/6886; C12N 15/1137; C07K 16/40; C12N 9/22; A61P 35/00

(57) ABSTRACT

Provided herein are methods and compositions for inhibiting p97, for the treatment of a cancer in a subject, or a symptom thereof. Upon treatment, the cancer, or a symptom thereof is reduced in the subject. Additionally, methods for measuring sensitivity of a subject to p97 inhibition, methods of assessing a pharmaceutical agent for p97 inhibition activity, and methods of assessing the effect of a pharmaceutical agent for p97 inhibition activity in a subject are provided herein.

### CROSS-REFERENCE TO RELATED APPLICATIONS

The present application claims the benefit of U.S. Provisional Application No. 63/276,905, filed on Nov. 8, 2021, which is hereby incorporated by reference in its entirety.

### STATEMENT REGARDING FEDERALLY SPONSORED R&D

This invention was made with government support under Grant No(s). NS102279 & NS100815 awarded by the National Institutes of Health. The government has certain rights in the invention.

### FIELD

Some embodiments described herein relate generally to p97 inhibition for treatment of cancer, such as a blood tumor, a solid tumor, a lymphoma, a myeloma, acute myeloid leukemia (AML), esophageal cancer, colon cancer, uterine cancer, or myelodysplastic syndrome (MDS). Other embodiments relate generally to measuring sensitivity of a subject to p97 inhibition, methods of assessing a pharmaceutical agent for p97 inhibition activity, and assessing the effect of a pharmaceutical agent on a subject.

### BACKGROUND

Protein homeostasis depends on regulation of protein degradation through the ubiquitin-proteasome system (UPS) and autophagy. Dysregulation of protein homeostasis is associated with cancer and the development of neurodegenerative disease [1]. Imbalances in protein synthesis caused by mutations in protein coding sequences and aneuploidy drive cancer cells toward stronger reliance on protein quality control (PQC) mechanisms. Interfering with PQC through proteasome inhibition has proven successful as an anticancer treatment, as indicated by FDA approval of two proteasome inhibitors (PI), bortezomib and carfilzomib [2]. In addition to the proteasome, p97 is another essential component of the protein homeostasis regulatory network and is implicated in several PQC pathways. p97 is a strong candidate as an alternative anticancer drug target in the PQC pathway [3]. p97 is an ATPase that removes misfolded proteins from the endoplasmic reticulum (ER) membrane for proteasomal degradation [4, 5] and also facilitates degradation of substrates embedded in other large structures, including mitochondria and chromosomes [6-8]. In addition to its role in regulating protein homeostasis, p97 is also involved in non-degradative pathways including Golgi and nuclear envelope reassembly and endosomal trafficking [9-12]. Recently, p97 was shown as required to clear damaged lysosomes [13, 14] and maintain lysosomal homeostasis [15].

### SUMMARY

In accordance with some embodiments described herein, methods for p97 inhibition for treatment of cancers, such as blood tumor, a solid tumor, a lymphoma, a myeloma, acute myeloid leukemia (AML), esophageal cancer, colon cancer, uterine cancer, or myelodysplastic syndrome (MDS) are provided.

Some embodiments provided herein relate to methods of measuring sensitivity of a subject to p97 inhibition. In some embodiments, the methods include identifying a subject having a cancer, or a symptom thereof; and measuring an expression profile of oncoproteins in a biological sample obtained from the subject. In some embodiments, the measured expression profile of the oncoproteins differs from a normal expression profile from a healthy subject. In some embodiments, the oncoproteins include cell cycle oncoproteins and/or oncoproteins of a Cyclin D1-CDK4/6-RB1-E2F1 pathway. In some embodiments, the oncoproteins of the Cyclin D1-CDK4/6-RB1-E2F1 pathway include Cyclin D1, CDK4, or ATF3.

In some embodiments, methods of measuring the expression profile include measuring expression of an E2F1 target gene. In some embodiments, the E2F1 target genes include RRM2, TK1, or DHFR. In some embodiments, the methods include administering an effective amount of an agent that inhibits p97 in the subject. In some embodiments, the cancer or a symptom thereof is reduced after the administering. In some embodiments, the cancer includes a blood tumor, a solid tumor, a lymphoma, a myeloma, acute myeloid leukemia (AML), esophageal cancer, colon cancer, uterine cancer, or myelodysplastic syndrome (MDS). In some embodiments, the agent that inhibits p97 is an inhibitory nucleic acid molecule, p97 binding antagonist, a genetic tool, and/or a small molecule inhibitor. In some embodiments, the cell cycle oncoproteins include Securin, MYC, Survivin, Emil, CDC20, Bub1, CDC25B, ORC6, GMNN, or RRM2. In some embodiments, the inhibitory nucleic acid molecule is an antisense nucleic acid. In some embodiments, the inhibitory nucleic acid molecule is a siRNA. In some embodiments, the inhibitory nucleic acid molecule is a shRNA. In some embodiments, the inhibitory nucleic acid molecule corresponds to or is complementary to at least a fragment of nucleic acid encoding p97. In some embodiments, the p97 binding antagonist inhibits the binding of p97 to its binding partners. In some embodiments, the p97 binding antagonist is an antibody against p97 or a fragment of p97. In some embodiments, the antibody is a monoclonal, polyclonal or an antibody fragment selected from the group consisting of Fab, Fab′-SH, Fv, scFv, and (Fab′)₂ fragments. In some embodiments, the genetic tool is a CRISPR/Cas9 system, a zinc finger nuclease system, a TALEN system, a homing endonucleases system or a meganuclease system. In some embodiments, the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

Some embodiments provided herein relate to methods of identifying a subject having a cancer with susceptibility to p97 inhibition. In some embodiments, the methods include detecting a level of a protein in a biological sample obtained from the subject. In some embodiments, the protein is Securin, CyclinD1, MYC, Survivin, Emil, CDC20, Bub1, CDC25B, ORC6, GMNN, RRM2, CDK4, ATF3, TK1, DHFR, or an ortholog thereof, or a combination thereof. In some embodiments, the methods further include detecting a presence, a genetic change and/or a level of the proteins. In some embodiments, the proteins are expressed differently and/or have a different genetic status in a sample obtained from a healthy subject.

Some embodiments provided herein relate to methods of improving, ameliorating, or treating a cancer. In some embodiments, the methods include detecting the genetic status, level, and/or expression of a cell cycle oncoprotein and/or a Cyclin D1-CDK4/6-RB1-E2F1 pathway oncoprotein in a subject; and comparing the genetic status, level, and/or expression of the cell cycle oncoprotein and/or the Cyclin D1-CDK4/6-RB1-E2F1 pathway oncoprotein to the genetic status, level and/or expression of a cell cycle oncoprotein and/or a Cyclin D1-CDK4/6-RB1-E2F1 pathway oncoprotein in a healthy subject. In some embodiments, detection of an abnormal genetic status or a high level in the subject relative to the normal subject indicates the presence of a cancer in the subject. In some embodiments, the methods further include administering to the subject an effective amount of an agent that inhibits p97 in the subject. In some embodiments, the agent that inhibits p97 is an inhibitory nucleic acid molecule, a p97 binding antagonist, a genetic tool, or a small molecule inhibitor. In some embodiments, the cancer or a symptom thereof is reduced after the administering. In some embodiments the Cyclin D1-CDK4/6-RB1-E2F1 pathway oncoprotein is Cyclin D1, CDK4, TK1, DHFR, or ATF3.

In some embodiments, the cancer is a blood tumor, a solid tumor, a lymphoma, a myeloma, acute myeloid leukemia (AML), esophageal cancer, colon cancer, uterine cancer, or myelodysplastic syndrome (MDS). In some embodiments, the cell cycle oncoproteins include Securin, MYC, Survivin, Emil, CDC20, Bub1, CDC25B, ORC6, GMNN, or RRM2. In some embodiments, the inhibitory nucleic acid molecule is an antisense nucleic acid. In some embodiments, the inhibitory nucleic acid molecule is a siRNA. In some embodiments, the inhibitory nucleic acid molecule is a shRNA. In some embodiments, the inhibitory nucleic acid molecule corresponds to or is complementary to at least a fragment of nucleic acid encoding p97. In some embodiments, the p97 binding antagonist inhibits the binding of p97 to its binding partners. In some embodiments, the p97 binding antagonist is an antibody against p97 or a fragment of p97. In some embodiments, the antibody is a monoclonal, polyclonal or an antibody fragment selected from the group consisting of Fab, Fab′-SH, Fv, scFv, and (Fab′)₂ fragments. In some embodiments, the genetic tool is CRISPR/Cas9 system, a zinc finger nuclease system, a TALEN system, a homing endonucleases system or a meganuclease system. In some embodiments, the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

Some embodiments provided herein relate to methods of assessing a pharmaceutical agent for p97 inhibition activity. In some embodiments, the methods include administering the pharmaceutical agent to a cancer cell; measuring expression levels of Cyclin D1, CDK4, Securin, MYC, Survivin, Emil, CDC20, Bub1, CDC25B, ORC6, GMNN, TK1, DHFR and/or RRM2; and comparing the measured expression levels to expression levels in cancer cells treated with control agents without p97 activity. In some embodiments, a reduction in said expression levels is used to assess p97 inhibition activity.

In some embodiments, the pharmaceutical agent is an inhibitory nucleic acid molecule, a p97 binding antagonist, a genetic tool, and/or a small molecule inhibitor. In some embodiments, the inhibitory nucleic acid molecule is an antisense nucleic acid. In some embodiments, the inhibitory nucleic acid molecule is a siRNA. In some embodiments, the inhibitory nucleic acid molecule is a shRNA. In some embodiments, the inhibitory nucleic acid molecule corresponds to or is complementary to at least a fragment of nucleic acid encoding p97. In some embodiments, the p97 binding antagonist inhibits the binding of p97 to its binding partners. In some embodiments, the p97 binding antagonist is an antibody against p97 or a fragment of p97. In some embodiments, the antibody is a monoclonal, polyclonal or an antibody fragment selected from the group consisting of Fab, Fab′-SH, Fv, scFv, and (Fab′)₂ fragments. In some embodiments, the genetic tool is CRISPR/Cas9 system, a zinc finger nuclease system, a TALEN system, a homing endonucleases system or a meganuclease system.

Some embodiments provided herein relate to methods of assessing the effect of a pharmaceutical agent on a subject. In some embodiments, the methods include administering a pharmaceutical agent to a cancer patient, and measuring the expression levels of oncoproteins Cyclin D1, CDK4, Securin, MYC, Survivin, Emil, CDC20, Bub1, CDC25B, ORC6, GMNN, TK1, DHFR and/or RRM2 from a biological sample from the patient. In some embodiments, a reduction in the expression levels is used as a pharmacodynamic marker in a subject to assess p97 inhibition activity by the pharmaceutical agent.

Some embodiments provided herein relate to methods of improving, ameliorating, or treating a cancer. In some embodiments, the methods include identifying a subject having a cancer, or a symptom thereof; and administering an effective amount of an agent that inhibits p97 in the subject. In some embodiments, the cancer or a symptom thereof is reduced after the administering.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 depicts a principal component analysis (PCA) on proteomic data from HCT116 cells treated with p97 shRNA (knockdown) or control shRNA showed that replicate samples had similar principal component (PC) scores.

FIGS. 2A-2G depict proteomic analysis of p97 inhibition with shRNA knockdown and pharmacological inhibitors. FIG. 2A shows a volcano plot displaying the proteomic changes following p97 knockdown in HCT116 cells, Log 2FC indicates the logarithm to the base 2 of fold change, n=3. FIG. 2B shows a Venn diagram displaying unfolded protein response (UPR) proteins that are differentially expressed (DE) caused by p97 KD. FIG. 2C depicts representative KEGG or Reactome pathways affected by p97 knockdown (KD) in HCT116 cells.

FIG. 2D shows a heatmap showing fold change in proteasome proteins, cell cycle related proteins and E2 ubiquitin-conjugating enzymes which are significantly dysregulated by p97 KD. FIG. 2E shows the percentage of overlapping DE proteins following p97 KD and p97 inhibitor treatment increases time-dependently. This percentage was calculated by dividing the number of overlapping proteins by the sum of all DE proteins identified by p97 KD and/or inhibitor treatment. HCT116 cells were treated with 4 μM UPCDC-30245, 2 μM CB-5083 or 4 μM of NMS-874 for the indicated time points, n=2. FIG. 2F shows functional enrichment analysis on proteins affected by both p97 KD and p97 inhibitors. FIG. 2G shows heatmap displays the fold change of DE proteins participating in the four cellular pathways affected by p97 inhibition.

FIGS. 3A-3E show validation of p97 knockdown in HCT116 cells. FIG. 3A depicts qPCR analysis of the RNA level of p97 in HCT116 treated with p97 shRNA or control. After 72 h induction with Doxycycline, the mRNA level of p97 was reduced by 79%. FIGS. 3B-3C depicts Western blot showing that the p97 protein level was reduced by 93.4% in the cytosol and by 68% in the nucleus following p97 KD. FIGS. 3D-3E depict Western blot detected the ER stress markers ATF3 was upregulated 7 at the protein level by p97 KD. FIG. 3F depicts quantification results of TCF11 detected by Western blot. For all experiments, n=3, *** indicates p<0.001, ** indicates p<0.01, * indicates p<0.05.

FIG. 4 depicts anti-proliferative effects CB-5083, NMS-873, UPCDC-30245 and MG132 on HCT116 cells. Cells were treated with the indicated inhibitor for 48 h.

FIG. 5 depicts PCA analysis on temporal proteomics data showed that the samples clustered into two groups according to treatment time. With the exception of the 6 h treatment with UPCDC-30245, the 2 h and 6 h treatments of CB-5083 and NMS-873 were grouped into one cluster while 8 h, 18 h and 24 h were grouped into another cluster.

FIGS. 6A-61I show comparison of the temporal proteomic profile resulting from treatment with p97 inhibitors and treatment with MG132. FIG. 6A shows principal component analysis (PCA) of tandem mass tag (TMT)-labeled proteomic data. HCT116 cells were treated with 1 μM MG132, 2 μM CB-5083 or 4 μM of NMS-874 for 6 h or 24 h, n=2. FIG. 6B shows Venn diagram displays the number of proteins that were dysregulated by NMS-873 and CB-5083 treatment. The DE proteins regulated by both p97 inhibitors can be separated into 3 groups. Group 1 contains 233 DE proteins that were only differentially expressed at 6 h (6 h, black rectangle), group 2 contains 349 DE proteins at both 6 h and 24 h (6 h & 24 h, blue rectangle), and group 3 contains 302 DE proteins differentially expressed only at 24 h (24 h, red rectangle). FIG. 6C shows hierarchical clustering of proteins regulated by both NMS-873 and CB-5083. M, C and N represents MG132, CB-5083 and NMS-873, respectively. FIG. 6D shows pathway analysis on proteins regulated both by MG132 and p97 inhibitors. FIG. 6E shows pathway analysis on proteins specifically downregulated by p97 inhibitors. FIG. 6F shows Venn diagram of the proteins upregulated by MG132, NMS-873 and CB-5083. FIG. 6G shows pathway analysis on proteins specifically upregulated by p97 inhibitors. (FIG. 6H), Log 2FC of four XBP1 activates genes which were significantly upregulated by CB-5083 (CB) and NMS-873 (NMS) after 24 h of treatment (p<0.05), but not by MG132 (MG).

FIG. 7A depicts cell cycle analysis of HCT116 cells after 24 h of treatments with DMSO, 1 μM of MG132 (MG) or 2 μM of CB-5083. FIG. 7B shows heatmap of autophagy related proteins which were dysregulated by both CB-5083 and NMS-873 after 6 h or 24 h of treatment. Data were from TMT labeling proteomics. M, C and N indicates MG132, CB-5083 and NMS-873 treatment, respectively. FIGS. 7C-7D show Western blotting validates the proteins identified from proteomic data. HCT116 cells were treated for 24 h with DMSO, 1 μM of MG132 (MG), 2 μM of CB-5083 (CB) or 4 μM of NMS-873 (NMS), n=3.

FIGS. 8A-8D depict identifying specific markers of p97 inhibition. FIGS. 8A-8B show a heatmap of proteins specifically downregulated by p97 inhibitors in HCT116 cells (FIG. 8A), and in HEK293 and U2OS cells (FIG. 8B). The log 2 fold change data of these proteins in HEK293 and U2OS cells was obtained from published results. Protein indicates proteomic data and RNA indicates RNAseq data. HEK293 cells were treated with p97 shRNA and NMS-873, U2OS cells were treated with NMS-873 and MG132. Blank indicates the protein was not detected. FIG. 8C depicts a heatmap showing proteins upregulated by both CB-5083 and NMS-873, but not by MG132, after 6 h or 24 h of treatment. Of the 210 upregulated overlapping DE proteins (p<0.05). FIG. 8D shows temporal proteomic profile of 6 potential p97 inhibition specific markers. Samples were treated with CB-5083, NMS-873, and MG132 (and data was collected from LFQ For FIGS. 8A-8C, M, C and N indicates MG132, CB-5083 and NMS-873 treatment respectively, shRNA represents p97 KD.

FIG. 9A depicts Western blotting indicates that both p97 inhibitors and MG132 reduced the levels of phosphorylated eIF4E-binding protein1 (phos-4EBP1). FIGS. 9B-9C show network of E2F1 (FIG. 9B) and cMyb (FIG. 9C). FIG. 9D shows CCNA2 and CDC6 which interact with E2F1 were dysregulated by CB-5083 (CB), NMS-873 (NMS) and MG132 (MG).

FIGS. 10A-10F depicts p97 inhibition impairs the transcriptional activity of E2F1 by downregulating the CCND1/CDK complex. FIG. 10A shows putative transcription factor binding sites (TFBSs) of the 33 proteins which were specifically downregulated by p97 inhibition. Data was analyzed using the g:Profiler website. Shading indicates the gene is a potential target of transcription factor c-Myb or E2F1. Blank indicates it is not a target gene. FIG. 10B depicts known regulatory network of the CCND1/CDK/RB1/E2F1 pathway. FIG. 10C shows log 2 fold change of the proteins regulating E2F1 function which were significantly affected by MG132 (MG), CB-5083 (CB) and NMS-873 (NMS) in TMT results. FIG. 10D shows dysregulation of E2F1 related proteins as confirmed by Western blot. HCT116 cells were treated with 1 μM MG132, 2 μM CB-5083 or 4 μM of NMS-874 for 6 h. FIGS. 10E and 10F show transcriptional activity of E2F1 measured using E2F1 reporter assay. HCT116 cells were transfected with pGL2-AN plasmid (Addgene: 20950) for 24 h. Then cells were plated in 384 well plate and incubated for 16 h. The luminescence were detected after 8 h treatment with MG132, CB-5083 or NMS-873. Data are shown as mean±SD, n=3. f, qRT-PCR analysis of ATF3, DHFR and CCND1 RNAs following treatment with MG132, CB-5083 or NMS-874. HCT116 cells were treated for 2, 6 and 24 h using the same concentration as proteomic and western blot assay (n=4).

FIGS. 11A-11B show the effect of p97 inhibitors and MG132 on E2F1 expression in HCT116 cells. FIG. 11A depicts qPCR analysis revealed the RNA level of E2F1 was strongly downregulated by MG132 and slightly downregulated by p97 inhibitors. FIG. 11B depicts Western blotting, indicating that E2F1 protein levels were not significantly affected by p97 inhibitors but were increased by MG132. For both qPCR and western blot assays, HCT116 cells were treated for 6 h with DMSO, 1 μM of MG132 (MG), 2 μM of CB-5083 (CB) or 4 μM of NMS-873 (NMS), n=2, **** indicates p<0.0001, *** indicates p<0.001, * indicates p<0.05 according to unpaired t-test.

FIGS. 12A-12C depicts Western blots detecting the effects of p97 inhibitors and MG132 on cell cycle proteins in HT29 and HCT116 cells. FIG. 12A shows HT29 cells treated for 6 h with DMSO, 1 μM of MG132, 2 μM of CB-5083 or 4 μM of NMS-873. FIG. 12B shows HCT116 cells treated with DMSO, 1 μM of MG132 (MG), 2 μM of CB-5083 (CB) or 1 μM of MG132 plus 2 μM of CB-5083 (CB+MG) for 1 h. Then, 50 μM of CHX was added and cells were collected at 0 h, 0.5 h, and 1 h. FIG. 12C depicts half-life of cyclin D1 in HCT116 cells. HCT116 cells were pre-treated with 5 μM of CB-5083 or DMSO for 30 minutes before adding 50 μM of CHX, and cells were collected at 0, 10, 20, 30 min (n=3), * indicates p<0.05 according to unpaired t-test.

FIGS. 13A-13G depict that p97 promotes the stability of cell-cycle oncoproteins. FIG. 13A shows qRT-PCR analysis of Myc, Securin, Emi and CDC20 mRNA levels. HCT116 cells were treated with 1 μM of MG132 or 2 μM of CB-5083 for 6 h, n=4. b-c, MG132 rescued CB-5083 mediated cyclin D1 downregulation at the protein level (FIG. 13B) but not at the mRNA level (FIG. 13C), while Baf A1 had no effect on cyclin D1. The concentration of MG132, CB-5083 and Baf A1 was 1 μM, 2 μM and 10 μM, respectively. Cells were treated for 6 hours, n=4, **** indicates p<0.001. FIGS. 13D-13E depict the half-life of Securin in HCT116 cells. HCT116 cells were treated with 1 μM of MG132, 2 μM of CB-5083, 1 μM of MG132 plus 2 μM of CB-5083 or DMSO. 50 μM of CHX was added immediately after compounds treatment, n=3, * indicates p<0.05. FIGS. 13F-13G show that the degradation of Securin was detected in the total lysate of HCT116 and HT29 cells. Cells were pretreated with 2 μM of MG132 for 1 h. The cells were harvested at 0 minutes, or the culture media was replaced with fresh media and DMSO, 2 μM of CB-5083 or 1 μM of MG132 added together with 50 μM of CHX for 60, 90, 120, 180 minutes, n=3, * indicates p<0.05, ** p<0.01. For FIGS. 13B-13G, D, B, M, C and N represents DMSO, Baf A1, MG132, CB-5083 and NMS-873, respectively. Data are shown as mean±SD. Statistical analysis was performed using one-way ANOVA.

FIG. 14A shows the expression of the eleven cell cycle genes that are specifically downregulated by p97/VCP inhibitors in different Gastrointestinal cancers. Batch adjusted normalized TCGA Pan-Cancer RNA-seq data was downloaded from the UCSC Xena Browser. Data represents log 2 of the ratio of the mean expression of tumor samples to the mean expression of matched normal samples. FIG. 14B shows boxplots depicting the mRNA expression of 10 out of the 11 proteins in colon cancer tumor tissue (N=275) and normal matched (N=41) using GEPIA web tool, * indicates p<0.05 and |log 2FC|>0.5.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

To assess whether p97 is a potential anticancer drug target, various groups have performed chemical screens in search of compounds that directly inhibit the ATPase activity of p97. Such screens identified a reversible ATP competitive inhibitor, DBeQ, [16] and two different allosteric p97 inhibitors, NMS-873 [17] and UPCDC-30245 [18-20]. Structure-activity-relationship (SAR) studies of DBeQ led to a collection of more potent and specific p97 inhibitors, including ML240 [21] and CB-5083 [22, 23]. CB-5083 strongly inhibited cancer cell proliferation, and was also efficacious in inhibiting tumor growth in mouse xenograft models implanted with HCT116 tumor cells [22]. CB-5083 entered phase I clinical trials for multiple myeloma and advanced solid tumors in 2015, highlighting p97 as a potential drug target in oncology. However clinical development was halted due to off-target effects [24]. p97 inhibitors remain a promising avenue however, and Cleave Therapeutics recently initiated a Phase 1 clinical trial of CB-5339 in patients with acute myeloid leukemia (AML) and myelodysplastic syndrome (MDS). The National Cancer Institute (NCI) is also evaluating CB-5339 for patients with solid tumors and lymphomas.

Without being bound by theory, current data indicate that p97 inhibitors are promising treatments in solid tumors where proteasome inhibitors are ineffective [25-28]. To facilitate the development of p97 inhibitors as potential therapeutic agents and help define their clinical application, it is necessary to dissect the mechanism of action (MOA) of p97 inhibitors and compare them with proteasome inhibitors (PIs). Moreover, identifying specific cellular markers is critical for both the drug discovery and development process, to help validate candidate drugs and quantify their effect.

### Definitions

Unless defined otherwise, technical, and scientific terms used herein have the same meaning as commonly understood when read in light of the instant disclosure by one of ordinary skill in the art to which the present disclosure belongs. For purposes of the present disclosure, the following terms are explained below.

The embodiments herein are generally disclosed using affirmative language to describe the numerous embodiments. Embodiments also include ones in which subject matter is excluded, in full or in part, such as substances or materials, method steps and conditions, protocols, or procedures.

In some embodiments, the numbers expressing quantities of ingredients, properties such as concentration, reaction conditions, and so forth, used to describe and claim certain embodiments described herein are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments are approximations, the numerical values set forth in the specific examples are reported as precisely as practicable. The numerical values presented in some embodiments may contain certain errors necessarily resulting from the standard deviation found in their respective testing measurements.

In some embodiments, the terms “a” and “an” and “the” and similar references used in the context of describing a particular embodiment described herein (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate embodiments and does not pose a limitation on the scope of the embodiments otherwise claimed. No language in the specification should be construed as indicating any non-claimed element essential to the practice of any of the embodiments described herein.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

Groupings of alternative elements or embodiments disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The term “inhibit” as used herein has its plain and ordinary meaning as understood in light of the specification and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. The term inhibit may not necessarily indicate a 100% inhibition. A partial inhibition may be realized.

The term “treatment” or “treating” means any administration of a compound or an agent according to the present disclosure to a subject having or susceptible to a condition or disease disclosed herein for the purpose of: 1) preventing or protecting against the disease or condition, that is, causing the clinical symptoms not to develop; 2) inhibiting the disease or condition, that is, arresting or suppressing the development of clinical symptoms; or 3) relieving the disease or condition that is causing the regression of clinical symptoms. In some embodiments, the term “treatment” or “treating” refers to relieving the disease or condition or causing the regression of clinical symptoms.

The term “effective amount” is meant as the amount of an agent required to reduce the symptoms of a disease relative to an untreated subject. The effective amount of agent(s) used to practice any of the embodiments described herein for therapeutic treatment of a cancer varies depending upon the manner of administration, the age, body weight, and general health of the subject. Ultimately, a physician or veterinarian will decide the appropriate amount and dosage regimen. Such amount is referred to as an “effective” amount.

Preferred embodiments are described herein, including the best mode known to the inventors for carrying out certain embodiments. Variations on those preferred embodiments will become apparent to those of ordinary skill in the art upon reading the foregoing description. It is contemplated that skilled artisans can employ such variations as appropriate, and embodiments can be practiced otherwise than specifically described herein. Accordingly, many embodiments include all modifications and equivalents of the subject matter recited in the claims appended hereto as permitted by applicable law. Moreover, any combination of the above-described elements in all possible variations thereof is encompassed unless otherwise indicated herein or otherwise clearly contradicted by context.

Furthermore, numerous references have been made to patents and printed publications throughout this specification. Each of the above cited references and printed publications are herein individually incorporated by reference in their entirety.

Embodiments disclosed herein are illustrative of the principles of the disclosure. Other modifications that can be employed can be within the scope of the disclosure. Thus, by way of example, but not of limitation, alternative configurations can be utilized in accordance with the teachings herein. Accordingly, embodiments are not limited to that precisely as shown and described.

### Inhibition of p97

Inhibition of p97 has been observed to effectively treat solid tumors whereas treatment with proteosome inhibitors is less effective. Accordingly, in some embodiments described herein, methods of treatment for cancer are provided. The methods can include administering an effective amount of an agent that promotes inhibition of p97 in the subject with a cancer. Following administration of an agent that promotes inhibition of p9′7, the cancer or a symptom thereof is reduced.

In some embodiments, methods of measuring sensitivity of a subject to p97 inhibition are provided, the methods include identifying a subject having a cancer, or a symptom thereof; and measuring expression profile of oncoproteins in a biological sample obtained from the subject. In some embodiments, the measured expression profile of the oncoproteins differs from a normal expression from a healthy subject.

In some embodiments, “measuring” can include assessing the expression profile of genes, including genes for oncoproteins, in a subject having cancer and healthy subjects. In some embodiments, measuring can further include comparing the expression profile of genes, including genes for oncoproteins, between subjects having cancer and healthy subjects. In some embodiments, “measuring” can include assessing the protein expression level of oncoproteins, in a subject having cancer and healthy subjects. In some embodiments, measuring can further include comparing the protein expression levels of oncoproteins, between subjects having cancer and healthy subjects. In some embodiments, measuring expression profile can include identification oncogenes in a subject that are making messenger RNA. In some embodiments, expression profile can be assessed by technologies including DNA microarrays, RNA sequencing, and qPCR, immunoassays, and mass spectrometry.

In some embodiments, “sensitivity to p97 inhibition” or “susceptibility to p97 inhibition” can indicate a reduction in cancer or cancer symptoms in a subject in response to p97 inhibitor treatment. In some embodiments reduction in cancer symptoms can include, but is not limited to, reduction in tumor volume, reduction in the number of cancer cells, partial remission, or complete remission.

In some embodiments, the biological sample can include but is not limited to, a blood sample, a tumor biopsy, cerebrospinal fluid sample, a saliva sample, a urine sample, or a bone marrow sample.

In some embodiments, a “normal” expression profile can correspond to the expression profile from a healthy subject, free from cancer.

In some embodiments, a different expression status between the measured expression of the oncoproteins and normal expression from a healthy subject can include overexpression, under expression, gain of function mutations, or loss of function mutations.

In some embodiments, p97 inhibition includes methods of directly inhibiting the ATPase activity of p97. In some embodiments, p97 inhibition includes methods of reducing transcription of p97 RNA. In some embodiments, p97 inhibition includes methods of reducing translation of p97 protein. In some embodiments, p97 inhibition includes methods of knocking out p97 genes.

In some embodiments, the methods further include administering an effective amount of an agent that inhibits p97 in the subject. In some embodiments, the cancer or a symptom thereof is reduced after the administering.

In some embodiments, expression status can be assessed by using Next Generation Sequencing (NGS), sequencing, Polymerase Chain Reaction (PCR), Loop-mediated isothermal amplification, Recombinase polymerase amplification, or antibody detection.

In some embodiments the oncoproteins include cell cycle oncoproteins and/or oncoproteins of a Cyclin D1-CDK4/6-RB1-E2F1 pathway. In some embodiments, the oncoproteins of the Cyclin D1-CDK4/6-RB1-E2F1 pathway include Cyclin D1, CDK4, or ATF3.

In some embodiments, the cell cycle oncoproteins include Securin, MYC, Survivin, Emil, CDC20, Bub1, CDC25B, ORC6, GMNN, or RRM2.

In some embodiments, various agents can be used to inhibit p97 in a subject that is in need of treatment for cancer. For example, a nucleic acid molecule can be used to inhibit p97. In some embodiments, an antagonist that binds and inhibits p97 can be used. As another example, small molecule inhibitors that inhibit p97 can be used. As still yet another example, a genetic tool can be used to inhibit p97.

In some embodiments, inhibition of p97 reduces a cancer or a symptom thereof. In some embodiments, the cancer includes blood tumor, a solid tumor, a lymphoma, a myeloma, AML, esophageal cancer, colon cancer, uterine cancer, or MDS. In some embodiments, the methods include administering a therapeutically effective amount of an agent that promotes inhibition of p97 to a subject in need thereof.

In some embodiments, the subject has a blood cancer. In some embodiments, the methods further include determining whether the subject has a blood cancer, and the effective amount of p97 inhibiting agent is administered if the subject has a blood cancer. In some embodiments, the subject has a solid tumor. In some embodiments, the methods further include determining whether the subject has a solid tumor, and the effective amount of p97 inhibiting agent is administered if the subject has a solid tumor. In some embodiments, the subject has a lymphoma. In some embodiments, the methods further include determining whether the subject has a lymphoma, and the effective amount of p97 inhibiting agent is administered if the subject has a lymphoma. In some embodiments, the subject has a myeloma. In some embodiments, the methods further include determining whether the subject has a myeloma, and the effective amount of p97 inhibiting agent is administered if the subject has a myeloma. In some embodiments, the subject has AML. In some embodiments, the methods further include determining whether the subject has AML, and the effective amount of p97 inhibiting agent is administered if the subject has AML. In some embodiments, the subject has esophageal cancer. In some embodiments, the methods further include determining whether the subject has esophageal cancer, and the effective amount of p97 inhibiting agent is administered if the subject has esophageal cancer. In some embodiments, the subject has colon cancer. In some embodiments, the methods further include determining whether the subject has colon cancer, and the effective amount of p97 inhibiting agent is administered if the subject has colon cancer. In some embodiments, the subject has uterine cancer. In some embodiments, the methods further include determining whether the subject has uterine cancer, and the effective amount of p97 inhibiting agent is administered if the subject has uterine cancer. In some embodiments, the subject has MDS. In some embodiments, the methods further include determining whether the subject has MDS and the effective amount of p97 inhibiting agent is administered if the subject has MDS.

In accordance with any of the embodiments described above, an effective amount of a nucleic acid molecule that corresponds to or is complementary to at least a fragment of nucleic acid encoding p97 is administered to inhibit p97. In accordance with any of the embodiments described above, the nucleic acid molecule is a siRNA. In some embodiments, the nucleic acid molecule is a shRNA. In accordance with any of the embodiments described above, the nucleic acid molecule is an antisense nucleic acid.

In accordance with any of the embodiments described above, an effective amount of antagonist that binds and inhibits p97 is administered. In accordance with any of the embodiments described above, the antagonist is an antibody against p97 or a fragment of p97. In accordance with any of the embodiments described above, the antibody is a monoclonal, polyclonal or an antibody fragment selected from the group consisting of Fab, Fab′-SH, Fv, scFv, and (Fab′)₂ fragments.

In accordance with any of the embodiments described above, a genetic tool is administered to inhibit p97. In accordance with any of the embodiments described above, the genetic tool to inhibit p97 is a CRISPR/Cas9 system. In accordance with any of the embodiments described above, the genetic tool to inhibit p97 is a zinc finger nuclease system. In accordance with any of the embodiments described above, the genetic tool to inhibit p97 is a TALEN system. In accordance with any of the embodiments described above, the genetic tool to inhibit p97 is a homing endonucleases system. In accordance with any of the embodiments described above, the genetic tool to inhibit p97 is a meganuclease system.

In accordance with any of the embodiments described above, a small molecule inhibitor is administered to inhibit p97. In accordance with any of the embodiments described above, the small molecule inhibitor to inhibit p97 is CB-5083. As used herein, the term CB-5083 has its ordinary meaning as understood in light of the specification and refers to a p97 AAA ATPase/VCP inhibitor that is orally bioavailable, and that selectively inhibits p9′7, and that has the chemical formula C₂₄H₂₃N₅O₂, with the chemical name of 1-(4-(benzylamino)-7,8-dihydro-5H-pyrano[4,3-d]pyrimidin-2-yl-2-methyl-1H-indole-4-carboxamide, and which has the chemical structure:

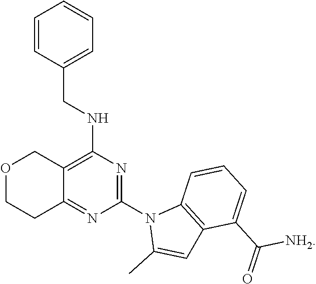 [SMILES: Cc1cc2c(C(N)=O)cccc2n1-c1nc2c(c(NCc3ccccc3)n1)COCC2]

In some embodiments, the small molecule inhibitor of p97 is CB-5083 or any functional salt, derivative, or analogue thereof.

In accordance with any of the embodiments described above, the small molecule inhibitor to inhibit p97 is NMS-873. As used herein, the term NMS-873 has its ordinary meaning as understood in light of the specification and refers to an allosteric p97 AAA ATPase/VCP inhibitor that is orally bioavailable, and that selectively inhibits p9′7, and that has the chemical formula C₂₇H₂₈N₄O₃S₂, with the chemical name of 3-[3-cyclopentylsulfanyl-5-[[3-methyl-4-(4-methylsulfonylphenyl)phenoxy]methyl]-1,2,4-triazol-4-yl]pyridine, and which has the chemical structure:

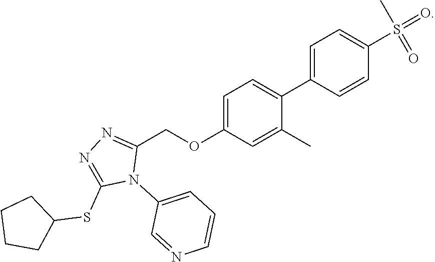 [SMILES: Cc1cc(OCc2nnc(SC3CCCC3)n2-c2cccnc2)ccc1-c1ccc(S(C)(=O)=O)cc1]

In some embodiments, the small molecule inhibitor of p97 is NMS-873 or any functional salt, derivative, or analogue thereof.

In accordance with any of the embodiments described above, the small molecule inhibitor to inhibit p97 is NMS-859. As used herein, the term NMS-859 has its ordinary meaning as understood in light of the specification and refers to a small molecule p97 AAA ATPase/VCP inhibitor, and that selectively inhibits p9′7, and that has the chemical formula C₁₅H₁₂ClN₃O₃S, with the chemical name of 2-chloro-N-(341,1-dioxidobenzo[d]isothiazol yl)amino)phenyl)acetamide, and which has the chemical structure:

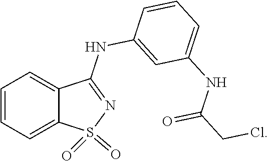 [SMILES: O=C(CCl)Nc1cccc(NC2=NS(=O)(=O)c3ccccc32)c1]

In some embodiments, the small molecule inhibitor of p97 is NMS-859 or any functional salt, derivative, or analogue thereof.

In accordance with any of the embodiments described above, the small molecule inhibitor to inhibit p97 is DBeQ. As used herein, the term DBeQ has its ordinary meaning as understood in light of the specification and refers to an ATP-competitive p97/VCP inhibitor, and that inhibits p97, and that has the chemical formula C₂₂H₂₀N₄, with the chemical name of N²,N⁴-Bis(phenylmethyl)-2,4-quinazolinediamine, and which has the chemical structure:

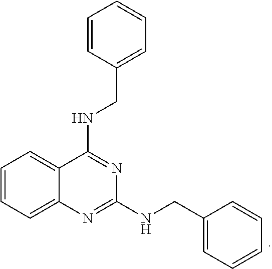 [SMILES: c1ccc(CNc2nc(NCc3ccccc3)c3ccccc3n2)cc1]

In some embodiments, the small molecule inhibitor of p97 is DBeQ or any functional salt, derivative, or analogue thereof.

In accordance with any of the embodiments described above, the small molecule inhibitor to inhibit p97 is MSC1094308. As used herein, the term MSC1094308 has its ordinary meaning as understood in light of the specification and refers to an allosteric p97 AAA ATPase/VCP inhibitor, and that inhibits p9′7, and that has the chemical formula C₂₉H₂₉F₃N₄, with the chemical name of N-((6-fluoro-2,3,4,9-tetrahydro-1H-carbazol-3-yl)methyl)-4,4-bis(4-fluorophenyl)butan-1-amine, and which has the chemical structure:

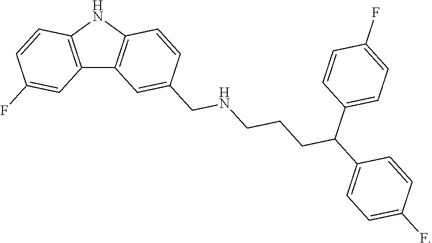 [SMILES: Fc1ccc(C(CCCNCc2ccc3[nH]c4ccc(F)cc4c3c2)c2ccc(F)cc2)cc1]

In some embodiments, the small molecule inhibitor of p97 is MSC1094308 or any functional salt, derivative, or analogue thereof.

In accordance with any of the embodiments described above, the small molecule inhibitor to inhibit p97 is ML240. As used herein, the term ML240 has its ordinary meaning as understood in light of the specification and refers to a p97 AAA ATPase/VCP inhibitor, and that selectively inhibits p97, and that has the chemical formula C₂₃H₂₀N₆O, with the chemical name of 2-(2-Amino-1H-benzimidazole-1-yl)-8-methoxy-N-(phenylethyl)-71-quinazolinamine, and which has the chemical structure:

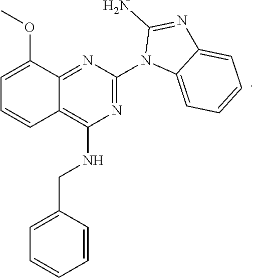 [SMILES: COc1cccc2c(NCc3ccccc3)nc(-n3c(N)nc4ccccc43)nc12]

In some embodiments, the small molecule inhibitor of p97 is ML240 or any functional salt, derivative, or analogue thereof.

In accordance with any of the embodiments described above, the small molecule inhibitor to inhibit p97 is p97-IN-1. As used herein, the term p97-IN-1 has its ordinary meaning as understood in light of the specification and refers to a p97/VCP inhibitor, and that selectively inhibits p97, and that has the chemical formula C₂₄H₂₄N₆O, and which has the chemical structure:

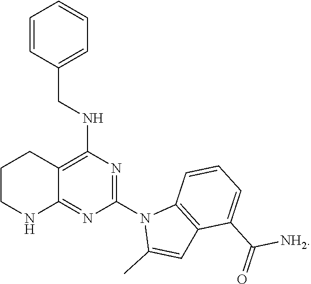 [SMILES: Cc1cc2c(C(N)=O)cccc2n1-c1nc2c(c(NCc3ccccc3)n1)CCCN2]

In some embodiments, the small molecule inhibitor of p97 is p97-IN-1 or any functional salt, derivative, or analogue thereof.

In accordance with any of the embodiments described above, the small molecule inhibitor to inhibit p97 is VCP/p97 inhibitor-1. As used herein, the term VCP/p97 inhibitor-1 has its ordinary meaning as understood in light of the specification and refers to a p97/VCP inhibitor, and that selectively inhibits p97, and that has the chemical formula C₂₄H₂₆BN₅O₄S, and which has the chemical structure:

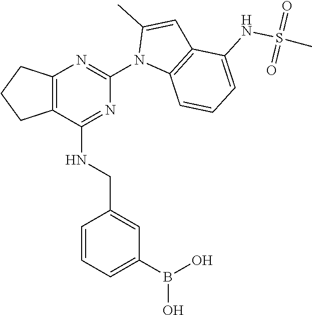 [SMILES: Cc1cc2c(NS(C)(=O)=O)cccc2n1-c1nc2c(c(NCc3cccc(B(O)O)c3)n1)CCC2]

In some embodiments, the small molecule inhibitor of p97 is VCP/p97 inhibitor-1 or any functional salt, derivative, or analogue thereof.

In accordance with any of the embodiments described above, the small molecule inhibitor to inhibit p97 is ML241 hydrochloride. As used herein, the term ML241 hydrochloride has its ordinary meaning as understood in light of the specification and refers to a p97 AAA ATPase/VCP inhibitor, and that selectively inhibits p9′7, and that has the chemical formula C₂₃H₂₅ClN₄O, with the chemical name of 2-(2H-benzo[b][1,4]oxazin-4(3H)-yl)-N-benzyl-5,6,7,8-tetrahydroquinazolin-4-amine hydrochloride, and which has the chemical structure:

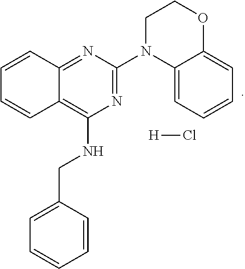

In some embodiments, the small molecule inhibitor of p97 is ML241 hydrochloride or any functional salt, derivative, or analogue thereof.

In accordance with any of the embodiments described above, the small molecule inhibitor to inhibit p97 is UPCDC-30245. As used herein, the term UPCDC-30245 has its ordinary meaning as understood in light of the specification and refers to an allosteric p97 AAA ATPase/VCP inhibitor, and that selectively inhibits p9′7, and that has the chemical formula C₂₈H₃₈FN₅, with the chemical name of 1-(3-(5-Fluoro-1H-indol-2-yl)phenyl)-N-(2-(4-isopropylpiperazin-1-yl)ethyl)piperidin-4-amine, and which has the chemical structure:

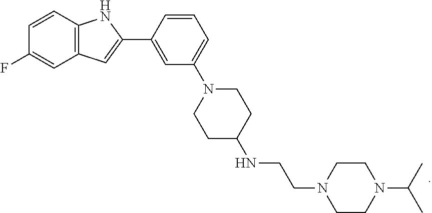 [SMILES: CC(C)N1CCN(CCNC2CCN(c3cccc(-c4cc5cc(F)ccc5[nH]4)c3)CC2)CC1]

In some embodiments, the small molecule inhibitor of p97 is UPCDC-30245 or any functional salt, derivative, or analogue thereof.

In some embodiments, inhibition of p97 increases or decreases the level or expression of genes associated with Cyclin D1-CDK4/6-RB1-E2F1 pathway. Examples of genes associated with Cyclin D1-CDK4/6-RB1-E2F1 pathway that are increased or decreased by inhibition of p97 include, but not limited to, E2F1, E2F2 CCND1, CDK4, CDK6, DHFR, TK1, RRM2, CDK2, CCNA2, CCNB1, and CCNB2.

In some embodiments, inhibition of p97 increases or decreases the level or expression of genes associated with progression of cancer. Examples of genes associated with progression of cancer that are increased or decreased by inhibition of p97 include, but not limited to, Cyclin D1, CDK4, Securin, MYC, Survivin, Emil, CDC20, Bub1, CDC25B, ORC6, GMNN, TK1, DHFR and/or RRM2.

In some embodiments, methods of identifying a subject having a cancer with susceptibility to p97 inhibition are provided. In some embodiments, the methods include detecting a level of a protein in a biological sample obtained from the subject. In some embodiments, the protein is Securin, CyclinD1, MYC, Survivin, Emil, CDC20, Bub1, CDC25B, ORC6, GMNN, RRM2, CDK4, ATF3, TK1, DHFR, or an ortholog thereof, or a combination thereof.

In some embodiments, the methods further include detecting a presence, a genetic change and/or a level of the proteins. In some embodiments, the proteins are expressed differently and/or have a different genetic status in a sample obtained from a healthy subject.

In some embodiments, a genetic change can include an increase in expression, a decrease in expression, a mutation in the gene for a protein, or a difference in the RNA splicing of a gene transcript. In some embodiments, genetic status can include levels of RNA expression, levels of protein expression, the nucleotide sequence of a gene, or the RNA sequence of a transcript. In some embodiments, expressed differently can include increased expression, decreased expression, a different isoform, a gain of function mutation, a loss of function mutation, or different post translational modification compared to a healthy subject.

In some embodiments, methods of improving, ameliorating, or treating a cancer are provided. In some embodiments, the methods include detecting the genetic status, level, and/or expression of a cell cycle oncoprotein and/or a Cyclin D1-CDK4/6-RB1-E2F1 pathway oncoprotein in a subject. In some embodiments, the methods further include comparing the genetic status, level, and/or expression of the cell cycle oncoprotein and/or the Cyclin D1-CDK4/6-RB1-E2F1 pathway oncoprotein to the genetic status, level and/or expression of a cell cycle oncoprotein and/or a Cyclin D1-CDK4/6-RB1-E2F1 pathway oncoprotein in a healthy subject. In some embodiments, detection of an abnormal genetic status and/or a high level in the subject relative to the normal subject indicates the presence of a cancer in the subject. In some embodiments, the methods further include administering to the subject an effective amount of an agent that inhibits p97 in the subject. In some embodiments, the agent that inhibits p97 is an inhibitory nucleic acid molecule, a p97 binding antagonist, a genetic tool, or a small molecule inhibitor. In some embodiments, the cancer or a symptom thereof is reduced after the administering. In some embodiments, the Cyclin D1-CDK4/6-RB1-E2F1 pathway oncoprotein is Cyclin D1, CDK4, TK1, DHFR, or ATF3.

In some embodiments, a high level of protein expression can include expression levels of a protein 10%, 20%, 30%, 40%, 50%, 60%, 70%, 80%, 90%, 100%, or an amount that is within a range defined by any two of the aforementioned values, or expression levels greater than 100% higher than expression levels found in samples from a healthy subject without cancer.

In some embodiments, methods of assessing a pharmaceutical agent for p97 inhibition activity are provided. In some embodiments, the methods include administering the pharmaceutical agent to a cancer cell, measuring expression levels of Cyclin D1, CDK4, Securin, MYC, Survivin, Emil, CDC20, Bub1, CDC25B, ORC6, GMNN, TK1, DHFR and/or RRM2, and comparing the measured expression levels to expression levels in cancer cells treated with control agents without p97 activity. In some embodiments, a reduction in said expression levels is used to assess p97 inhibition activity.

In some embodiments the cancer cell can be a commercially available cancer cell line. In some embodiments, the cancer cell can be a primary cancer cell derived from a subject with cancer. In some embodiments, the control agent without p97 inhibition activity can be a vehicle such as dimethyl sulfoxide (DMSO). In some embodiments treatments with the pharmaceutical agent can include dosages of 1 μM, 2 μM, 3 μM, 4 μM, 5 μM, 10 μM, within a range defined by any two of the aforementioned values. In some embodiments, treatments with the pharmaceutical agent can include timepoints of 1, 2, 4, 6, 8, 10, 12, 14, 16, 18, 20, 22, 24, 48, 72 hours, or within a range defined by any two of the aforementioned values.

In some embodiments, methods of assessing the effect of a pharmaceutical agent on a subject are provided. In some embodiments, the methods include administering a pharmaceutical agent to a cancer patient, and measuring the expression levels of oncoproteins Cyclin D1, CDK4, Securin, MYC, Survivin, Emi 1, CDC20, Bub1, CDC25B, ORC6, GMNN, TK1, DHFR and/or RRM2 from a biological sample from the patient. In some embodiments, a reduction in said expression levels is used as a pharmacodynamic marker in a subject to assess p97 inhibition activity by the pharmaceutical agent.

In some embodiments, assessing the effect of a pharmaceutical agent on a subject can include determining oncoprotein expression levels from a patient sample following administration of the agent, and measuring reduction of cancer or the symptoms thereof in a subject following administration of the pharmaceutical agent. In some embodiments, assessment of the effect of a pharmaceutical agent on a subject can include determining oncoprotein expression levels and measuring reduction of cancer or the symptoms thereof during ongoing administration of the pharmaceutical agent. In some embodiments, dose and duration of the pharmaceutical agent treatment can be adjusted based upon assessment of the effect of the pharmaceutical agent on a subject.

In some embodiments, methods of improving, ameliorating, or treating a cancer are provided. In some embodiments, the methods include identifying a subject having a cancer, or a symptom thereof; and administering an effective amount of an agent that inhibits p97 in the subject. In some embodiments, the cancer or a symptom thereof is reduced after the administering.

As described herein, inhibiting p97 can treat, inhibit, or ameliorate cancer symptoms. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### Dosage and Administration of p97 Inhibitors for Treating Cancer

Doses of p97 inhibitors can be readily determined for a given subject based on their body mass, disease type and state, and desired aggressiveness of treatment. In some embodiments, inhibitors of p97 are administered at a dose ranging from 1 mg/kg to 200 mg/kg, such as 1, 2, 3, 4, 5, 10, 15, 20, 25, 30, 35, 40, 45, 50, 55, 60, 65, 70, 75, 80, 85, 90, 95, 100, 110, 120, 130, 140, 150, 160, 170, 180, 190, or 200 mg/kg, or an amount within a range defined by any two of the aforementioned values. The composition may be administered twice daily, once daily, twice weekly, once weekly, or once monthly, or at a frequency within a range defined by any two of the aforementioned values.

In accordance with embodiments described herein, inhibitors of p97 can be administered by any suitable route of administration. Without limitation, the inhibitors of p97 can be administered to the subject via oral administration, rectum administration, transdermal administration, intranasal administration, or inhalation. In some embodiments, the inhibitors of p97 are administered to the subject orally. In some embodiments, the inhibitors of p97 can be administered by injection or in the form of a tablet, capsule, patch or a drink.

Pharmaceutically acceptable carriers are ones which are nontoxic to the cell or mammal being exposed thereto at the dosages and concentrations employed. Pharmaceutically acceptable carriers in accordance with methods and uses and compositions herein can include, but not limited to, organic or inorganic, solid or liquid excipients which is suitable for the selected mode of application such as oral application or injection and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also include one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers.

### Methods of Treating a Cancer

Described herein are methods of treatment of cancer. In some embodiments, the sensitivity to p97 inhibition in a subject in need of treatment for cancer is determined. In some embodiments, provided are methods for treating cancer in a subject that is amenable to treatment by inhibiting p97.

Various methods can be used to inhibit p97 in a subject and reduce the cancer, or a symptom thereof. For example, an ATP-competitor can be used to inhibit the enzyme activity of p97. In some embodiments, treatment with an allosteric p97 inhibitor can be used to inhibit p97.

In some embodiments, provided are methods for treating cancer. In some embodiments, the cancer is a blood tumor, a solid tumor, a lymphoma, a myeloma, AML, esophageal cancer, colon cancer, uterine cancer, or MDS. In some embodiments, the methods include administering a therapeutically effective amount of an agent that promotes inhibition of p97, to a subject in need thereof.

In various embodiments, the method is for treating cancer, including inhibition of p97 in a subject who is in need of treatment for cancer, thereby treating the subject. In various embodiments, the method is for treating a blood cancer, including inhibition of p97 in a subject who is in need of treatment for a blood cancer, thereby treating the subject. In various embodiments, the method is for treating a solid tumor, including inhibition of p97 in a subject who is in need of treatment for a solid tumor, thereby treating the subject. In various embodiments, the method is for treating a lymphoma, including inhibition of p97 in a subject who is in need of treatment for a lymphoma thereby treating the subject. In various embodiments, the method is for treating a myeloma including inhibition of p97 in a subject who is in need of treatment for a myeloma, thereby treating the subject. In various embodiments, the method is for treating AML, including inhibition of p97 in a subject who is in need of treatment for AML, thereby treating the subject. In various embodiments, the method is for treating esophageal cancer, including inhibition of p97 in a subject who is in need of treatment for esophageal cancer, thereby treating the subject. In various embodiments, the method is for treating colon cancer, including inhibition of p97 in a subject who is in need of treatment for colon cancer, thereby treating the subject. In various embodiments, the method is for treating uterine cancer, including inhibition of p97 in a subject who is in need of treatment for uterine cancer, thereby treating the subject. In various embodiments, the method is for treating MDS, including inhibition of p97 in a subject who is in need of treatment for MDS, thereby treating the subject.

In some embodiments as described above, the methods further include identifying a subject who would benefit from inhibiting mutant p97. The methods can include administering an effective amount of an agent to inhibit p97. In some embodiments, the subject is in need of p97 inhibition, and following administration of p97 inhibiting agent, the cancer, or a symptom thereof is reduced in the subject.

In some embodiments, the methods further include identifying a subject who would benefit from inhibiting p97. The methods can include administering an effective amount of an agent to inhibit p97. In some embodiments, the subject is in need of p97 inhibition, and following administration of a p97 inhibiting agent, dysregulated Cyclin D1-CDK4/6-RB1-E2F1 pathway is regulated.

In some embodiments, the methods further include identifying a subject who would benefit from inhibiting p97. The methods can include administering an effective amount of an agent to inhibit p97. In some embodiments, the subject is in need of p97 inhibition, and following administration of a p97 inhibiting agent, the level or expression of genes associated with progression of cancer are regulated. Examples of genes associated with progression of cancer that are increased or decreased by inhibition of p97 include, but not limited to, Securin, MYC, Survivin, Emil, CDC20, Bub1, CDC25B, ORC6, GMNN, or RRM2.

In some embodiments, the p97 inhibiting agent is administered to the subject until a cancer, or a symptom thereof in the subject is reduced. Optionally, the p97 inhibiting agent is administered to the subject after a cancer, or a symptom thereof in the subject is reduced, for example to solidify or maintain the subject free of cancer.

As described herein, inhibiting mutant p97 can treat, inhibit, or ameliorate cancer, or a symptom thereof. As disclosed herein, amelioration is used in a broad sense to refer to at least a reduction in the magnitude of a parameter, e.g., symptom, associated with the pathological condition being treated. In some embodiments, the method can completely inhibit, e.g., prevent from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

Embodiments provided herein are further described in the following enumerated embodiments.

1. A method of measuring sensitivity of a subject to p97 inhibition, the method comprising: identifying a subject having a cancer, or a symptom thereof; and measuring an expression profile of oncoproteins in a biological sample obtained from the subject, wherein the measured expression profile of the oncoproteins differs from a normal expression profile from a healthy subject; wherein the oncoproteins comprise cell cycle oncoproteins or oncoproteins of a Cyclin D1-CDK4 or 6-RB1-E2F1 pathway; and wherein the oncoproteins of the Cyclin D1-CDK4 or 6-RB1-E2F1 pathway comprise Cyclin D1, CDK4, or ATF3.

2. The method of embodiment 1, wherein measuring the expression profile comprises measuring expression of an E2F1 target gene.

3. The method of embodiment 2, wherein the E2F1 target gene comprises RRM2, TK1, or DHFR.

4. The method of embodiment 1, wherein the cancer comprises a blood tumor, a solid tumor, a lymphoma, a myeloma, acute myeloid leukemia (AML), esophageal cancer, colon cancer, uterine cancer, or myelodysplastic syndrome (MDS).

5. The method of embodiment 1, wherein the cell cycle oncoproteins comprise Securin, MYC, Survivin, Emil, CDC20, Bub1, CDC25B, ORC6, GMNN, or RRM2.

6. The method of embodiment 1, further comprising administering an effective amount of an agent that inhibits p97 in the subject, wherein the cancer or a symptom thereof is reduced after the administering.

7. The method of embodiment 6, wherein the agent that inhibits p97 is an inhibitory nucleic acid molecule, a p97 binding antagonist, a genetic tool, or a small molecule inhibitor.

8. The method of embodiment 7, wherein the inhibitory nucleic acid molecule is an antisense nucleic acid.

9. The method of embodiment 7, wherein the inhibitory nucleic acid molecule is a siRNA.

10. The method of embodiment 7, wherein the inhibitory nucleic acid molecule is a shRNA.

11. The method of embodiment 7, wherein the inhibitory nucleic acid molecule corresponds to or is complementary to at least a fragment of nucleic acid encoding p97.

12. The method of embodiment 7, wherein the p97 binding antagonist inhibits the binding of p97 to its binding partners.

13. The method of embodiment 12, wherein the p97 binding antagonist is an antibody against p97 or a fragment of p97.

14. The method of embodiment 13, wherein the antibody is a monoclonal, polyclonal or an antibody fragment selected from the group consisting of Fab, Fab′-SH, Fv, scFv, and (Fab′)₂ fragments.

15. The method of embodiment 7, wherein the genetic tool is selected from the group consisting of a CRISPR/Cas9 system, a zinc finger nuclease system, a TALEN system, a homing endonucleases system, and a meganuclease system.

16. The method of embodiment 7, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

17. A method of identifying a subject having a cancer with susceptibility to p97 inhibition, the method comprising: detecting a level of a protein in a biological sample obtained from the subject, wherein the protein is Securin, CyclinD1, MYC, Survivin, Emil, CDC20, Bub1, CDC25B, ORC6, GMNN, RRM2, CDK4, ATF3, TK1, DHFR, or an ortholog thereof, or a combination thereof.

18. The method of embodiment 17, further comprising detecting a presence, a genetic change, or a level of the protein, wherein the protein is expressed differently or has a different genetic status than a sample obtained from a healthy subject.

19. A method of improving, ameliorating, or treating a cancer, the method comprising: detecting the genetic status, level, or expression of a cell cycle oncoprotein or a Cyclin D1-CDK4 or 6-RB1-E2F1 pathway oncoprotein in a subject; comparing the genetic status, level, or expression of the cell cycle oncoprotein or the Cyclin D1-CDK4 or 6-RB1-E2F1 pathway oncoprotein to the genetic status, level or expression of a cell cycle oncoprotein or a Cyclin D1-CDK4 or 6-RB1-E2F1 pathway oncoprotein in a healthy subject, wherein detection of an abnormal genetic status or a high level in the subject relative to the normal subject indicates the presence of a cancer in the subject; and administering to the subject an effective amount of an agent that inhibits p97 in the subject, wherein the agent that inhibits p97 is an inhibitory nucleic acid molecule, a p97 binding antagonist, a genetic tool, or a small molecule inhibitor; wherein the cancer or a symptom thereof is reduced after the administering, and wherein the Cyclin D1-CDK4 or 6-RB1-E2F1 pathway oncoprotein is Cyclin D1, CDK4, TK1, DHFR, or ATF3.

20. The method of embodiment 19, wherein the cancer comprises a blood tumor, a solid tumor, a lymphoma, a myeloma, acute myeloid leukemia (AML), esophageal cancer, colon cancer, uterine cancer, or myelodysplastic syndrome (MDS).

21. The method of embodiment 19, wherein the cell cycle oncoprotein is Securin, MYC, Survivin, Emil, CDC20, Bub1, CDC25B, ORC6, GMNN, or RRM2.

22. The method of embodiment 19, wherein the inhibitory nucleic acid molecule is an antisense nucleic acid.

23. The method of embodiment 19, wherein the inhibitory nucleic acid molecule is a siRNA.

24. The method of embodiment 19, wherein the inhibitory nucleic acid molecule is a shRNA.

25. The method of embodiment 19, wherein the inhibitory nucleic acid molecule corresponds to or is complementary to at least a fragment of nucleic acid encoding p97.

26. The method of embodiment 19, wherein the p97 binding antagonist inhibits the binding of p97 to its binding partners.

27. The method of embodiment 26, wherein the p97 binding antagonist is an antibody against p97 or a fragment of p97.

28. The method of embodiment 27, wherein the antibody is a monoclonal, polyclonal or an antibody fragment selected from the group consisting of Fab, Fab′-SH, Fv, scFv, and (Fab′)₂ fragments.

29. The method of embodiment 19, wherein the genetic tool is selected from the group consisting of a CRISPR/Cas9 system, a zinc finger nuclease system, a TALEN system, a homing endonucleases system, and a meganuclease system.

30. The method of embodiment 19, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

31. A method of assessing a pharmaceutical agent for p97 inhibition activity, comprising: administering the pharmaceutical agent to a cancer cell; measuring expression levels of Cyclin D1, CDK4, Securin, MYC, Survivin, Emil, CDC20, Bub1, CDC25B, ORC6, GMNN, TK1, DHFR and/or RRM2; and comparing the measured expression levels to expression levels in cancer cells treated with control agents without p97 activity; wherein a reduction in said expression levels is used to assess p97 inhibition activity.

32. The method of embodiment 31, wherein the pharmaceutical agent is an inhibitory nucleic acid molecule, a p97 binding antagonist, a genetic tool, or a small molecule inhibitor.

33. The method of embodiment 32, wherein the inhibitory nucleic acid molecule is an antisense nucleic acid.

34. The method of embodiment 32, wherein the inhibitory nucleic acid molecule is a siRNA.

35. The method of embodiment 32, wherein the inhibitory nucleic acid molecule is a shRNA.

36. The method of embodiment 32, wherein the inhibitory nucleic acid molecule corresponds to or is complementary to at least a fragment of nucleic acid encoding p97.

37. The method of embodiment 32, wherein the p97 binding antagonist inhibits the binding of p97 to its binding partners.

38. The method of embodiment 37, wherein the p97 binding antagonist is an antibody against p97 or a fragment of p97.

39. The method of embodiment 38, wherein the antibody is a monoclonal, polyclonal or an antibody fragment selected from the group consisting of Fab, Fab′-SH, Fv, scFv, and (Fab′)₂ fragments.

40. The method of embodiment 32, wherein the genetic tool is selected from the group consisting of a CRISPR/Cas9 system, a zinc finger nuclease system, a TALEN system, a homing endonucleases system, and a meganuclease system.

41. A method of assessing the effect of a pharmaceutical agent on a subject, the method comprising: administering a pharmaceutical agent to a cancer patient; and measuring the expression levels of oncoproteins Cyclin D1, CDK4, Securin, MYC, Survivin, Emil, CDC20, Bub1, CDC25B, ORC6, GMNN, TK1, DHFR and/or RRM2 from a biological sample from the patient, wherein a reduction in said expression levels is used as a pharmacodynamic marker in a subject to assess p97 inhibition activity by the pharmaceutical agent.

42. The method of embodiment 41, wherein the pharmaceutical agent is an inhibitory nucleic acid molecule, p97 binding antagonist, a genetic tool, or a small molecule inhibitor.

43. The method of embodiment 42, wherein the inhibitory nucleic acid molecule is an antisense nucleic acid.

44. The method of embodiment 42, wherein the inhibitory nucleic acid molecule is a siRNA.

45. The method of embodiment 42, wherein the inhibitory nucleic acid molecule is a shRNA.

46. The method of embodiment 42, wherein the inhibitory nucleic acid molecule corresponds to or is complementary to at least a fragment of nucleic acid encoding p97.

47. The method of embodiment 42, wherein the p97 binding antagonist inhibits the binding of p97 to its binding partners.

48. The method of embodiment 47, wherein the p97 binding antagonist is an antibody against p97 or a fragment of p97.

49. The method of embodiment 48, wherein the antibody is a monoclonal, polyclonal or an antibody fragment selected from the group consisting of Fab, Fab′-SH, Fv, scFv, and (Fab′)₂ fragments.

50. The method of embodiment 42, wherein the genetic tool is selected from the group consisting of a CRISPR/Cas9 system, a zinc finger nuclease system, a TALEN system, a homing endonucleases system, and a meganuclease system.

51. A method of improving, ameliorating, or treating a cancer, the method comprising: identifying a subject having a cancer, or a symptom thereof; and administering an effective amount of an agent that inhibits p97 in the subject, wherein the cancer or a symptom thereof is reduced after the administering.

### EXAMPLES

Some aspects of the embodiments discussed above are disclosed in further detail in the following examples, which are not in any way intended to limit the scope of the present disclosure.

### Example 1: Proteomic Profiling and Analysis of p97 Inhibition and Knockdown

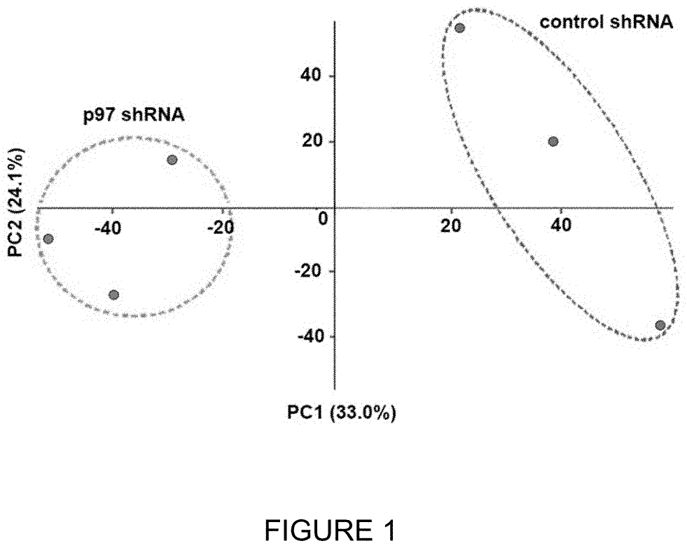

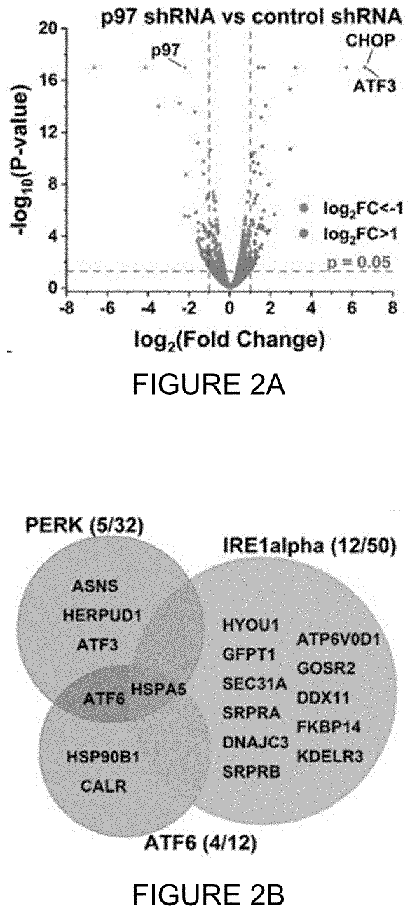

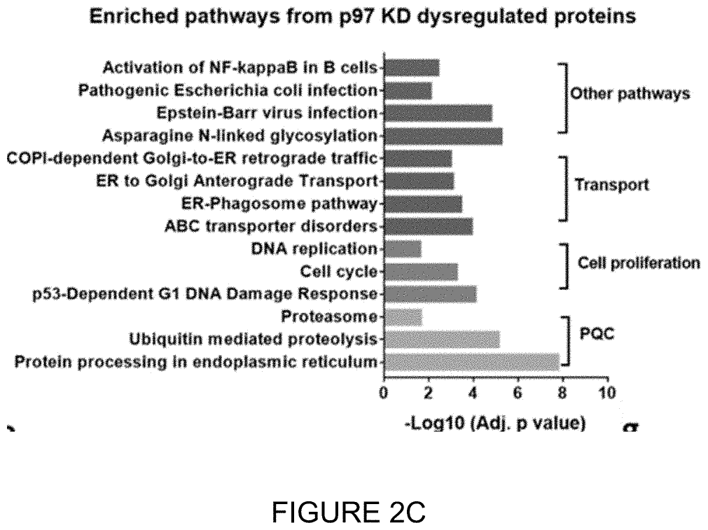

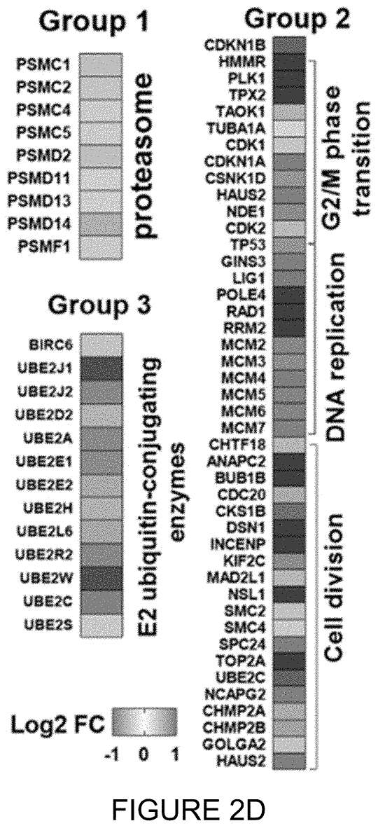

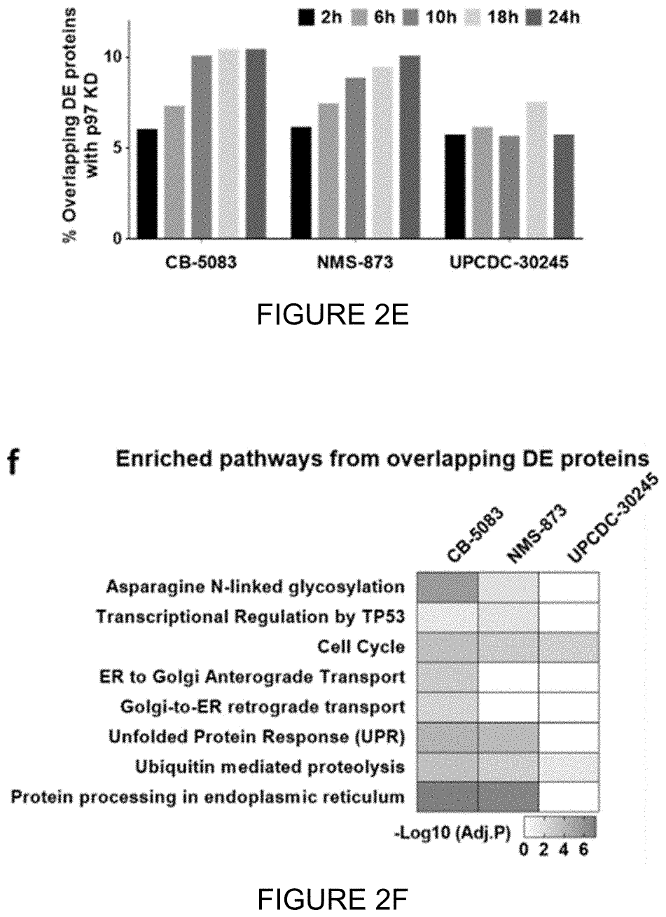

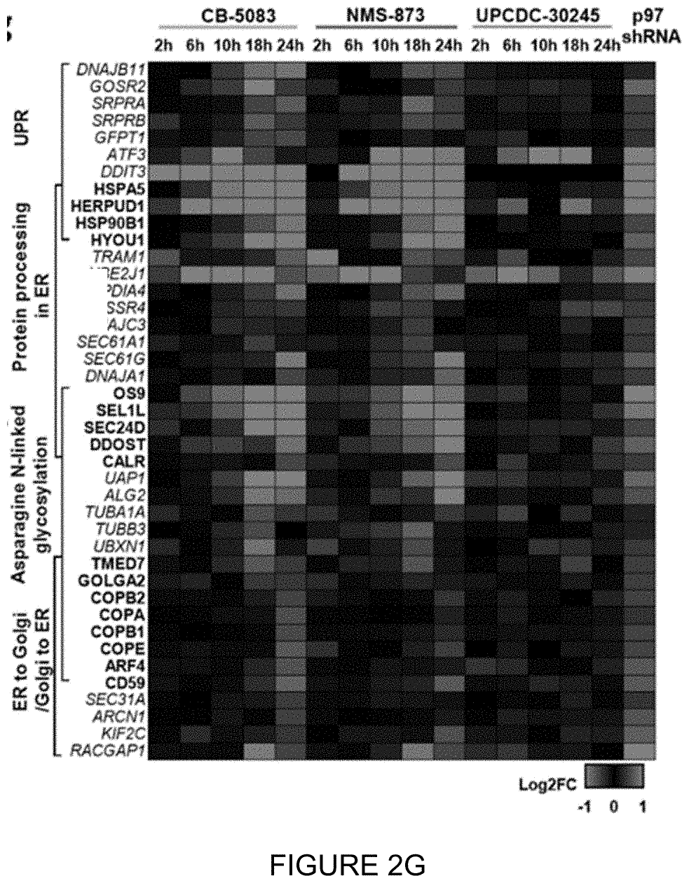

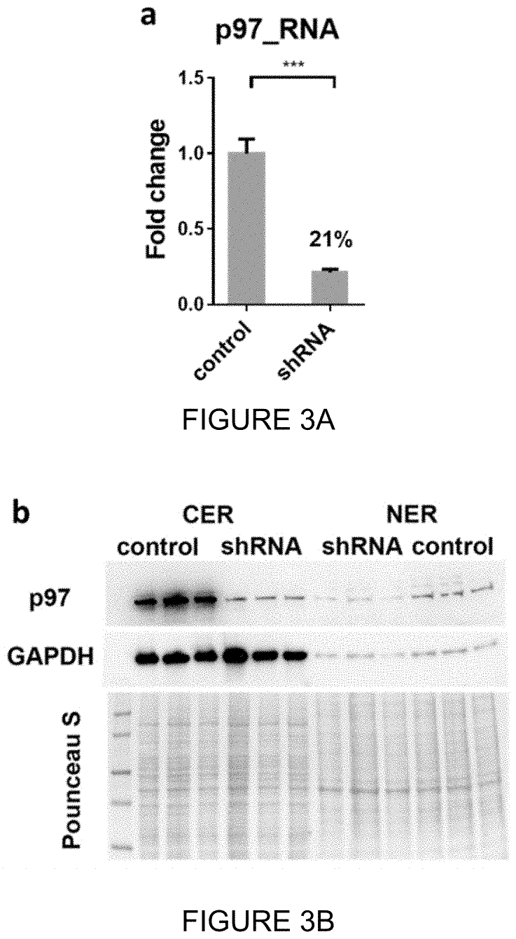

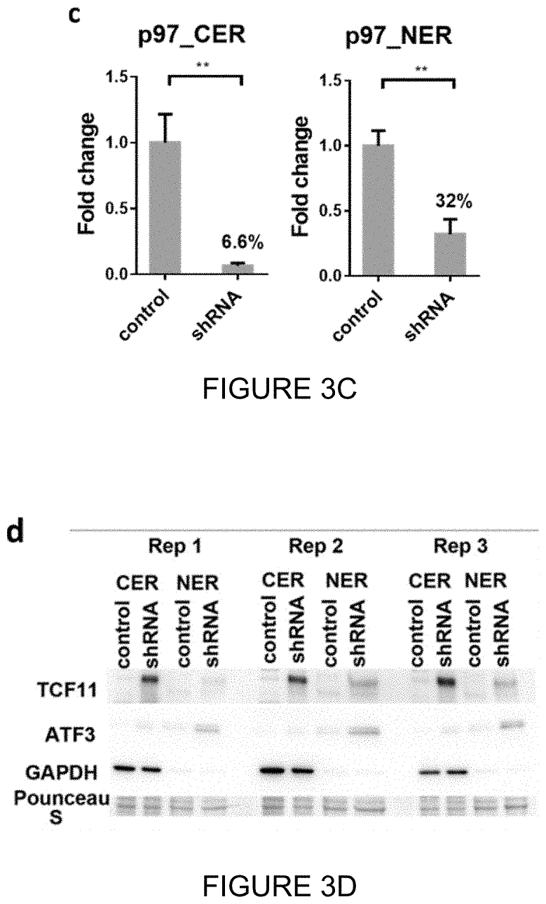

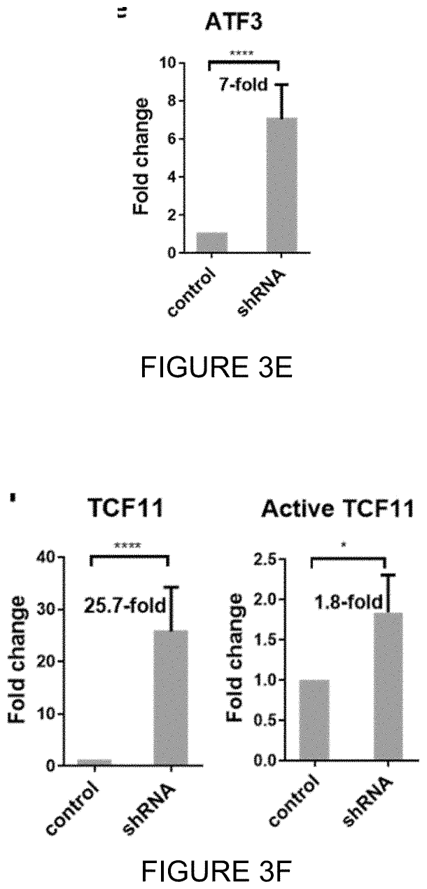

The first orally bioavailable p97 inhibitor, CB-5083, was originally developed using xenografts-derived from HCT116 colon cancer cells [22]. HCT116 cells were chosen to evaluate the effect of p97 inhibition, and proteomic analysis was used with label-free quantification (LFQ), which provides the deepest coverage of unperturbed proteomes [29], to comprehensively measure the functional impact of p97 knockdown. From a 2 h gradient run on nano-LC coupled with Eclipse mass spectrometry (MS), 6884 proteins were identified and quantified. Principal component analysis (PCA) showed that replicate samples had similar principal component scores (FIG. 1), indicating that the biological replicates correlated well with each other. By performing differential expression (DE) analysis, 410 proteins were identified that were significantly upregulated, and 356 proteins that were significantly downregulated (p-value<0.05) in p97 KD HCT116 cells. DE analysis confirmed that p97 was downregulated by 78% and that ATF3 and DDIT3 (CHOP) were upregulated more than 10-fold (FIGS. 2A-2G). The change in these proteins was consistent with western blot analysis (FIGS. 3A-3F).

Identified DE proteins were compared to components of the three key unfolded protein response (UPR) pathways, PERK, ATF6 and IRE1a. p97 knockdown lead to DE of 5 proteins in the PERK pathway, 4 proteins in ATF6 pathway and 12 proteins in IRE1a pathway. These proteins were all upregulated in p97 KD HCT116 cells, indicating that in some embodiments, p97 depletion in HCT116 cells activates all three UPR pathways (FIG. 2B). Functional enrichment analysis on all DE proteins revealed that multiple well-known p9′7-associated functions were affected by p97 KD, such as PQC, cell proliferation, and transport (FIG. 2C). In addition, the data showed that the Asparagine N-linked glycosylation pathway was affected by p97 KD. Asparagine N-linked glycosylation is an important form of protein post-translational modification (PTM) and is dependent on ER quality control (ERQC) and ER-to-Golgi transport [30, 31]. The dysregulation of this pathway indicates that in some embodiments, p97 participates in maintaining PTMs. Multiple proteins in cellular pathways linked to p97 function were upregulated upon p97 KD, including proteasome components and E2 ubiquitin-conjugating enzymes (E2s; FIG. 2D). In some embodiments, the upregulation of proteasome components and E2s compensates for reduced proteolytic activity due to p97 KD. A significant increase in nuclear levels of active TCF11 in p97 KD cells was detected (FIGS. 3D and 3F). This change contributes to transcriptional upregulation of proteasome components, consistent with previous reports [32, 33]. The majority of cell cycle related proteins were downregulated by p97 KD, including the MCM complex (FIG. 2D). This finding is consistent with the lower proliferation rate of p97 KD cells and with the essential role of p97 in DNA replication [34, 35].

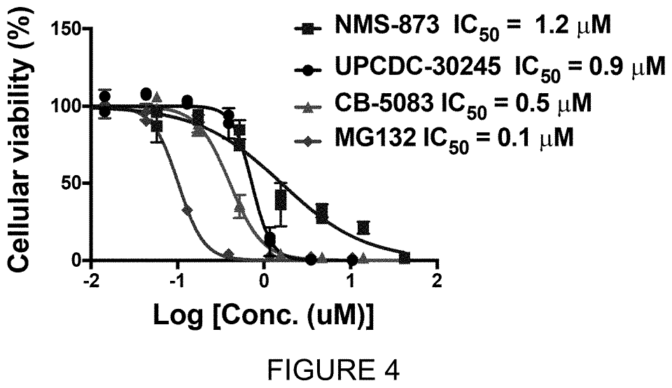

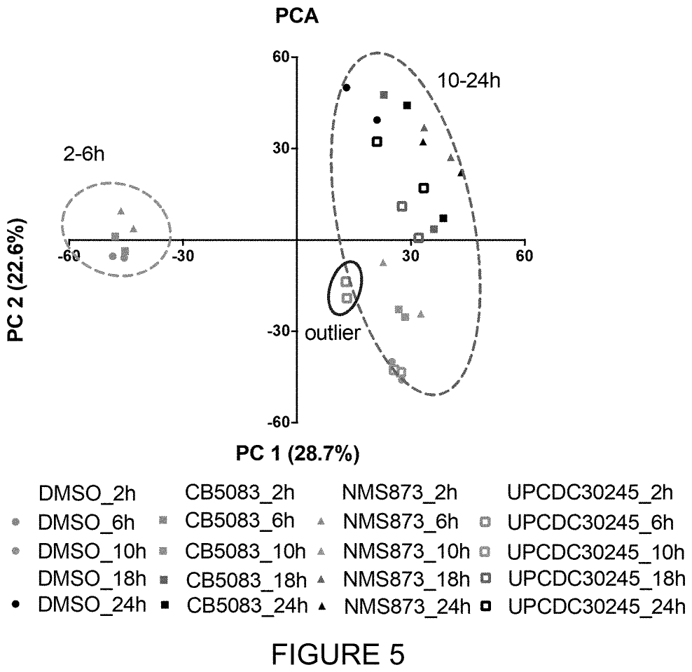

To explore the temporal proteomic profiles of p97 inhibition, HCT116 cells were treated with DMSO, and p97 inhibitors (CB-5083, NMS-873 or UPCDC-30245) for 2 h, 6 h, 10 h, 18 h, or 24 h in duplicate. To allow consistent comparison between inhibitors, a treatment concentration that is 4-fold of the IC50 concentration was chosen to aim for a similar cellular potency and avoid indirect effects caused by cell death (FIG. 4). 7703 proteins were identified from 40 samples. PCA on the temporal proteomics profiles showed that 2 h and 6 h treatment of CB-5083 and the NMS-873 treatment samples grouped in one cluster while 8 h, 18 h and 24 h grouped into another cluster (FIG. 5). Therefore, 6 h and 24 h represent reasonable timepoints to probe early and late responses to p97 inhibition. To compare the proteomic profile of p97 shRNA KD to that of different p97 inhibitors, DE proteins identified through p97 KD were compared with those identified under different inhibitor treatments (p<0.05) at each time point. With the exception of UPCDC-30245, the percentage of overlapping DE proteins increased with treatment duration (FIG. 2E). The proteomic profile of p97 KD was most similar to later timepoints following pharmacological p97 inhibition since p97 depletion by shRNA takes 72 h. To reveal the cellular effects common to both p97 KD and p97 inhibitors, overlapping DE proteins across different timepoints for each inhibitor treatment were combined. Functional enrichment analysis on each of these overlapping protein sets revealed that overlapping DE proteins from CB-5083 and NMS-873 treatment are linked to the same cellular pathways affected by p97 KD (FIG. 2F). Interestingly, it was found that DE proteins that are affected by both p97 KD and UPCDC-30245 are not linked to pathways typically impacted by p97 inhibition, such as protein processing in the ER, UPR and asparagine N-linked glycosylation (FIG. 2F). UPR proteins and those linked to protein processing in the ER were all upregulated by CB-5083, NMS-873 and p97 KD, but not by UPCDC-30245 (FIG. 2G). These data indicate that in some embodiments, the MOA of CB-5083 and NMS-873 in HCT116 cells is similar to p97 KD but is distinct from the MOA of UPCDC-30245.

### Example 2: Comparative Proteomic Analysis of p97 and Proteasome Inhibition Reveals Differential Molecular Mechanisms

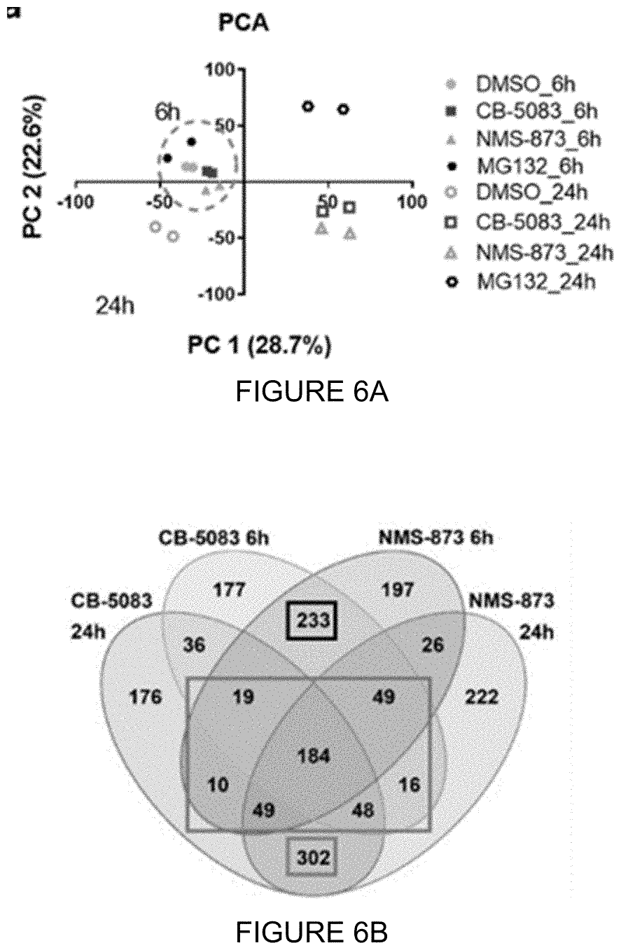

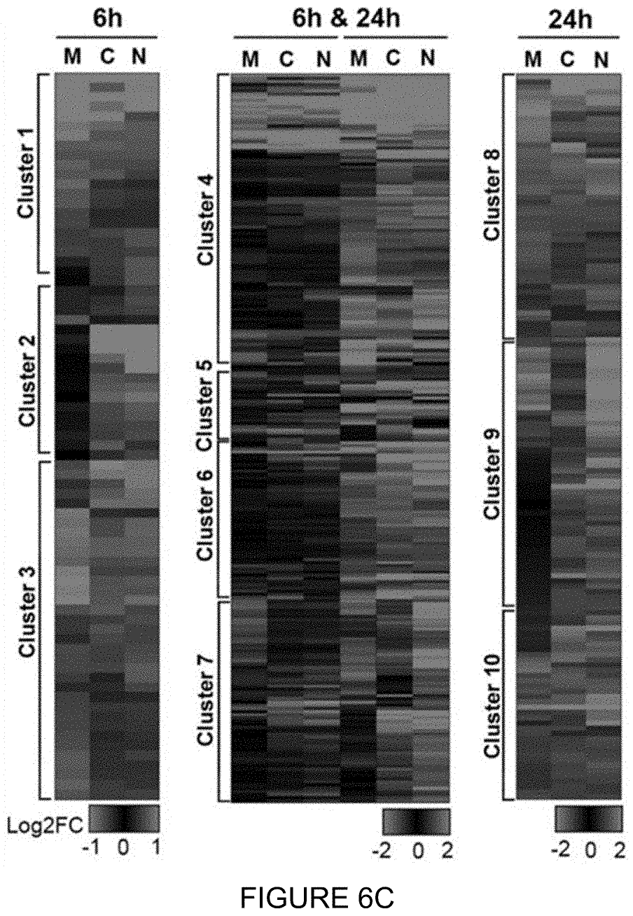

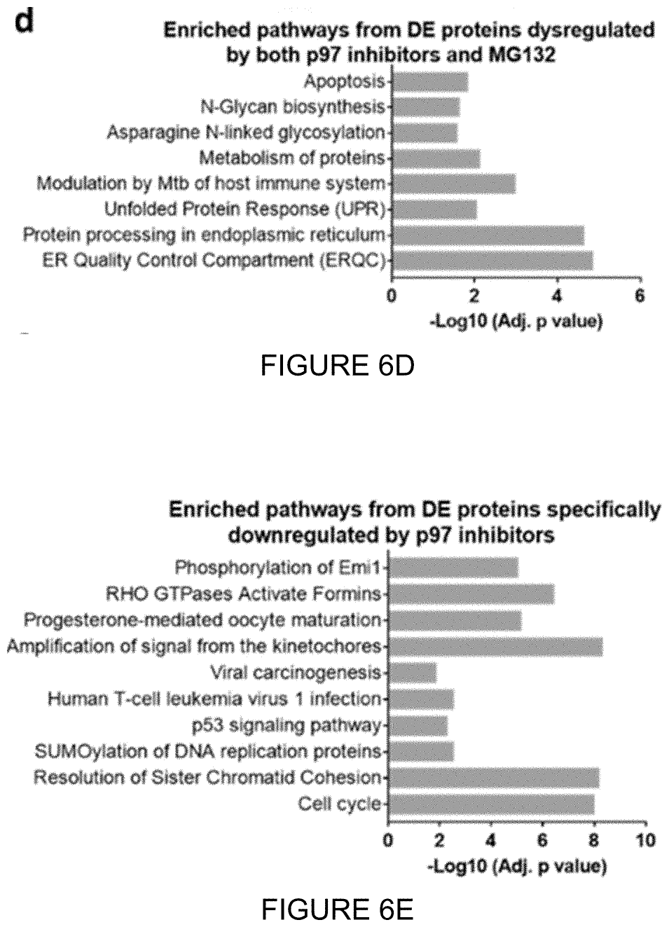

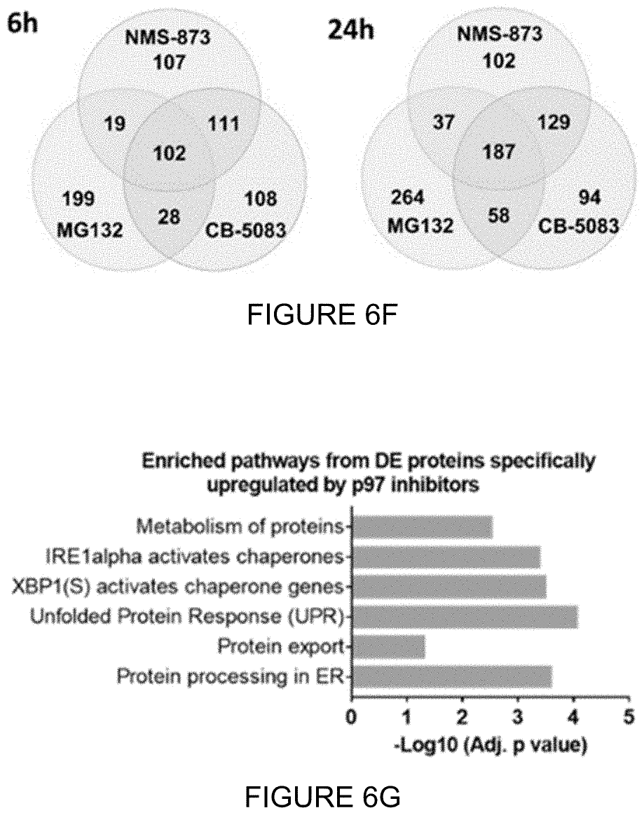

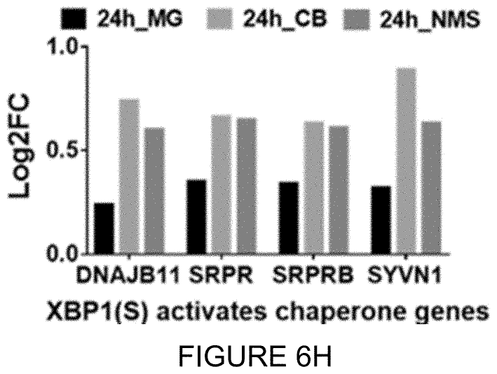

As a step toward understanding the differential effects of inhibiting p97 and the proteasome in cancer treatment, the effect of inhibiting p97 was compared to proteasome inhibition using tandem mass tag (TMT) 16-plexed labelling. This technique allowed for capture of alterations in the proteome after exposure to MG132 (proteasome inhibitor) and p97 inhibitors (CB-5083 and NMS-873). Considering the previous results, 6 h and 24 h treatments were chosen as representative of early (6 h) and late (24 h) responses to inhibitors. Samples were pooled together, fractionated into 8 fractions using high pH reverse chromatography, and analyzed using the highly accurate RTS-SPS-MS3 method [29, 36]. 7942 proteins were identified, of which 6956 proteins were quantified across all 16 samples. PCA showed 6 h samples clustered separately from 24 h samples, consistent with LFQ results, and confirmed that treatment time contributed to substantial changes in protein expression (FIG. 6A).

To identify the proteomic changes common to the different p97 inhibitors, DE analysis was performed and identified DE proteins (p<0.05) affected in both CB-5083 and NMS-873 treated samples (FIG. 6B). As shown in FIG. 6B, the overlapping DE proteins were divided into 3 groups (6 h, 6 h & 24 h, or 24 h) and performed hierarchical clustering on those proteins which were significantly dysregulated by p97 inhibitors in respective samples with a threshold of |log 2FC|>0.5 (FIG. 6C). Clusters 1, 4, and 8 contained proteins upregulated by both p97 inhibitors and MG132. Clusters 3, 6, and 10 contained proteins downregulated by both p97 inhibitors and MG132. Clusters 2, 7, and 9 contained proteins downregulated by p97 inhibitors but not MG132. Since p97 acts upstream of the proteasome and plays an important role in UPS, as expected the majority of DE proteins affected by p97 inhibitors were regulated similarly by MG132 (clusters 1, 4, 8, 3, 6 and 10). Functional enrichment analysis on the DE proteins dysregulated by both p97 inhibitors and MG132 identified pathways related to UPS, such as ERQC, protein processing in ER and UPR (FIG. 6D).

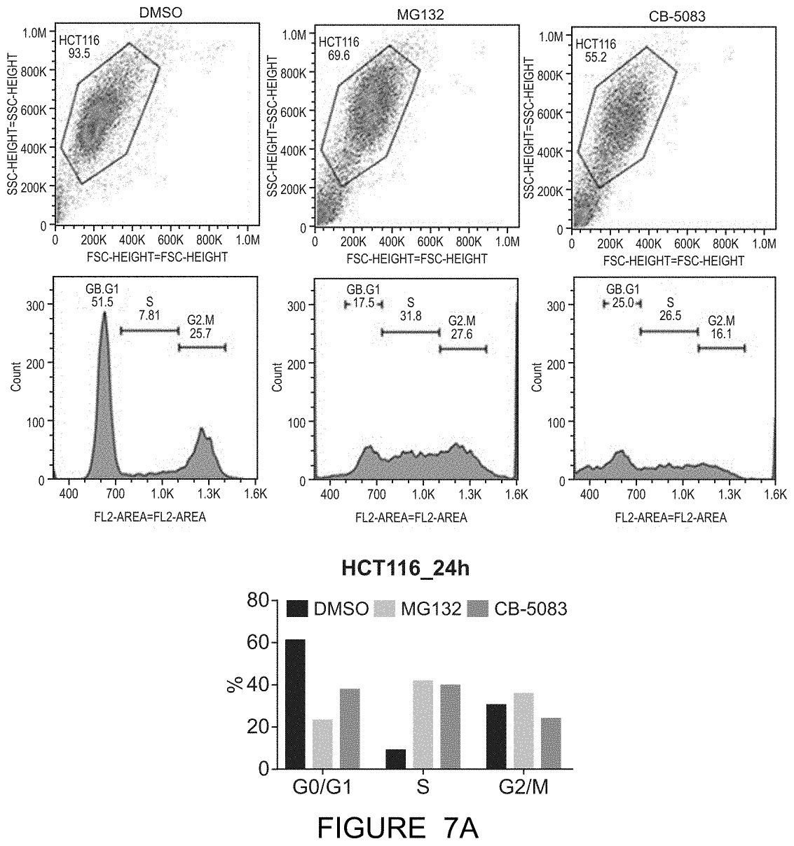

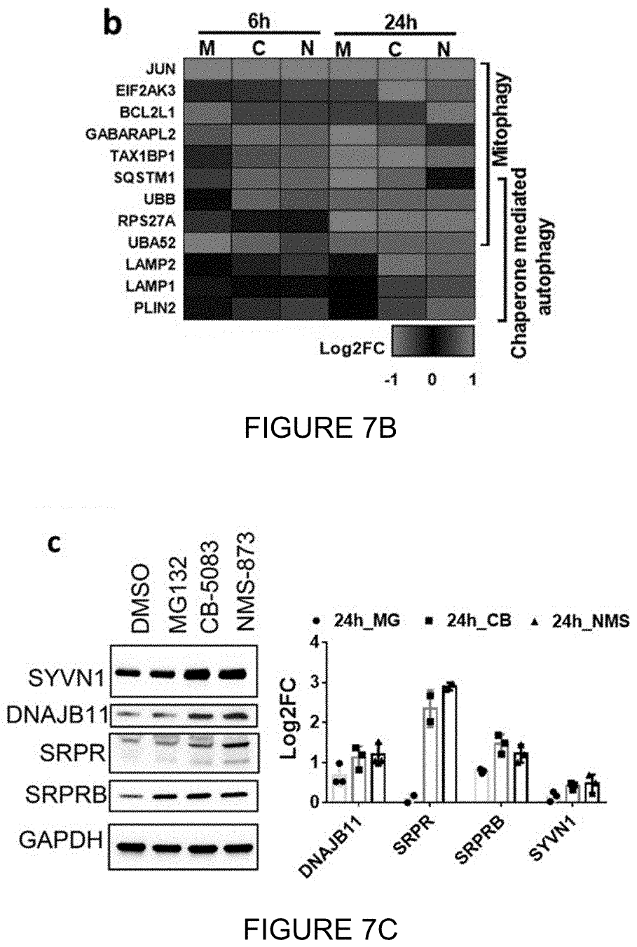

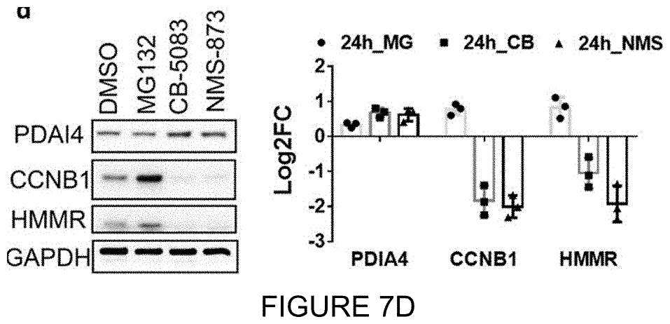

Next, the proteins downregulated by p97 inhibitors but not by MG132 (clusters 2, 7, and 9) were examined. Using hierarchical clustering, 121 proteins were identified that were specifically downregulated by p97 inhibitors. These proteins are involved in the p53 signaling pathway, cell cycle, virus infection and RHO GTPase-related pathways (FIG. 6E). To compare the cell cycle effects induced by a p97 inhibitor to that induced by a proteasome inhibitor, the DNA content was measured in both HCT116 cells after treatments with CB-5083 or MG132 for 24 h (FIG. 7A) and showed a reduced distribution in the G0/G1 phase when treated with CB-5083 or MG132. Compared to MG132, CB-5083 is less potent in reducing the distribution of cells in G0/G1 phase and in increasing the distribution of cells in G2/M phase.

Moreover, it was found that the autophagy related proteins BCL2L1, GABARAPL2 (ATG8C), TAX1BP1 and p62 (SQSTM1) were downregulated at 6 h and upregulated at 24 h by p97 inhibitors in cluster 5. Both TAX1BP1 and p62 were upregulated by MG132 at 6 h and 24 h (FIG. 7B). No proteins specifically upregulated by p97 inhibitors using hierarchical clustering were identified. Therefore, a Venn diagram was used to examine upregulated proteins (p<0.05, |log 2FC|>0.3) identified from MG132, NMS-873 and CB-5083 treatment to identify proteins that were specifically upregulated by p97 inhibitors (FIG. 6F). From the Venn diagram, 210 DE proteins were identified which were specifically upregulated by p97 inhibitors but not by MG132 after combining 6 h and 24 h data (FIG. 6F). Functional enrichment analysis on these proteins revealed that p97 inhibition had a different effect on protein processing in the ER and UPR relative to MG132 treatment. In particular, p97 inhibition showed higher potency in activating the IREα and XBP1 pathways (FIG. 6G). Four XBP1 activated proteins, DNAJB11, SRPR, SRPRB and SYVN1 were significantly upregulated by p97 inhibitors but not by MG132 after 24 h treatment (FIG. 6H). The upregulation of these four proteins was further validated by western blot (FIG. 7C). These results are consistent with reported findings that CB-5083 is more potent at activating the XBP1 pathway than PIs in models of multiple myeloma [37]. Taken together, these results validate that comparative proteomic analysis can reveal the differential effect of p97 versus proteasome inhibition.

Overall, these results indicate that in some embodiments, p97 inhibition impacts specific cellular pathways that are unaffected by proteasome inhibition.

### Example 3: Identifying Specific Protein Markers of p97 Inhibition

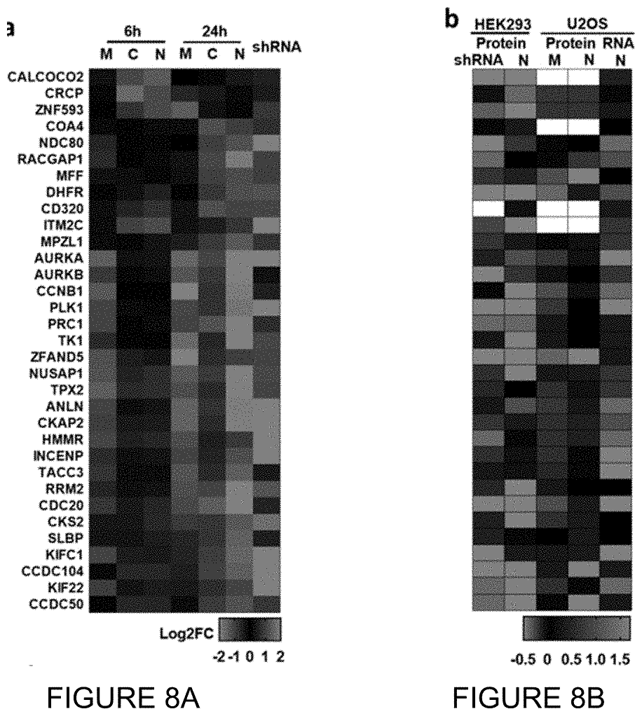

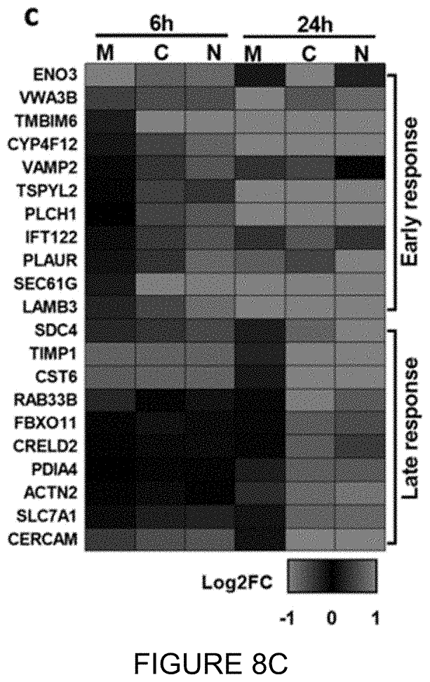

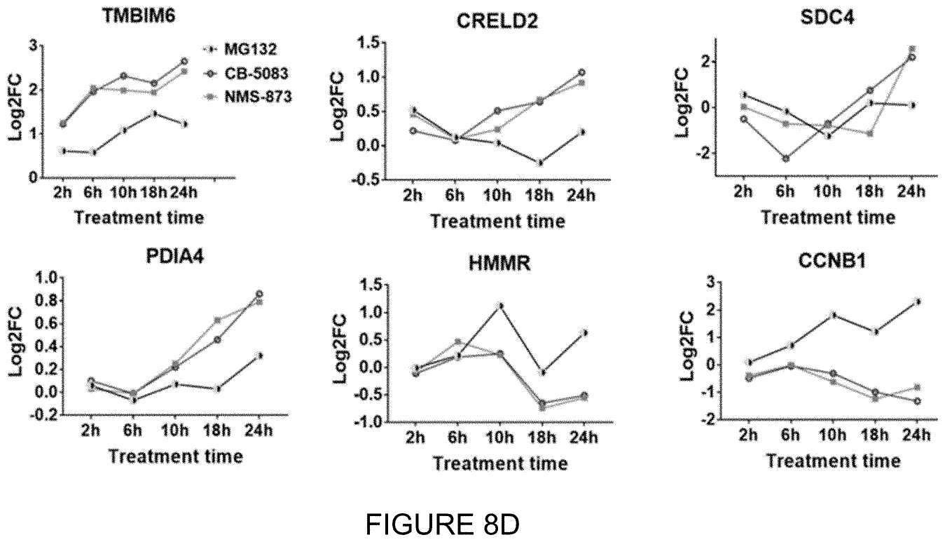

In order to validate the 121 downregulated proteins that are specifically affected by p97 inhibitors, these proteins were compared with the proteins that were downregulated by p97 KD 33 overlapping proteins were found (FIG. 8A). Comparing the present data to two published studies in HEK293 and U2OS cells revealed that the 33 proteins are also downregulated by p97 KD and NMS-873 treatment in other datasets (FIG. 8B) [38, 39]. This comparison demonstrated these 33 protein levels are regulated similarly by p97 inhibition in HCT116, HEK293, and U2OS cells. The 210 upregulated proteins were further analyzed and it was found that the 21 most significantly upregulated proteins were affected by p97 inhibitors but not by MG132 (FIG. 8C). Further comparison of the fold change calculated from TMT experiments with that from LFQ results for the 21 upregulated and 33 downregulated proteins and in samples treated with p97 inhibitors or MG132 was performed. Six out of the 54 proteins were also identified in the LFQ runs and showed time-dependent changes in response to p97 inhibitors (FIG. 8D). Of the six proteins, the regulation of CCNB1, PAID4 and HMMR were validated through western blot analysis (FIG. 7D) and Benjamini-Hochberg adjusted p-values for FDR less than 0.05 at least in one condition for all six proteins. These results indicate that these 6 proteins are affected by p97 but not proteasome inhibition, and are potential markers that distinguish these two treatments.

### Example 4: p97 Inhibition Blocks E2F1-Mediated Transcription Via Downregulation of the CCND-CDK4/6 Complex

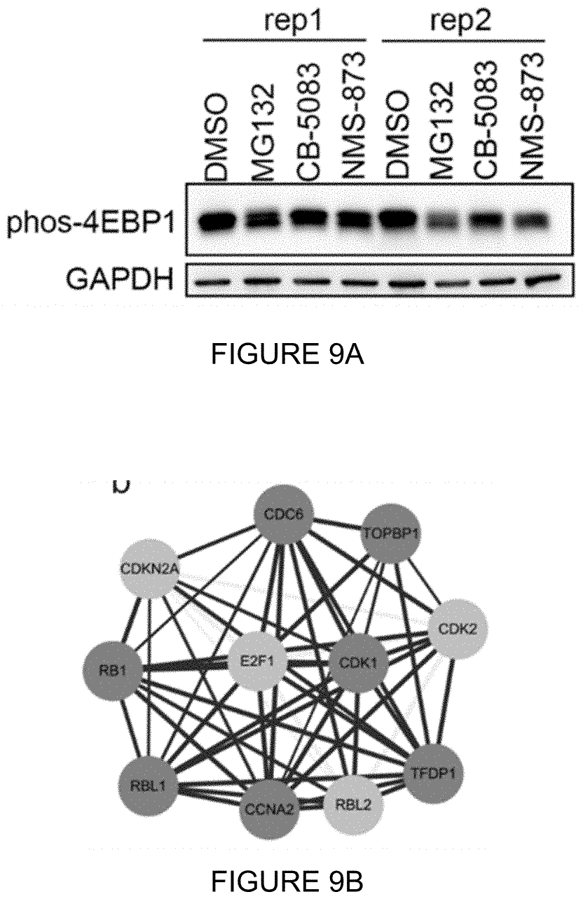

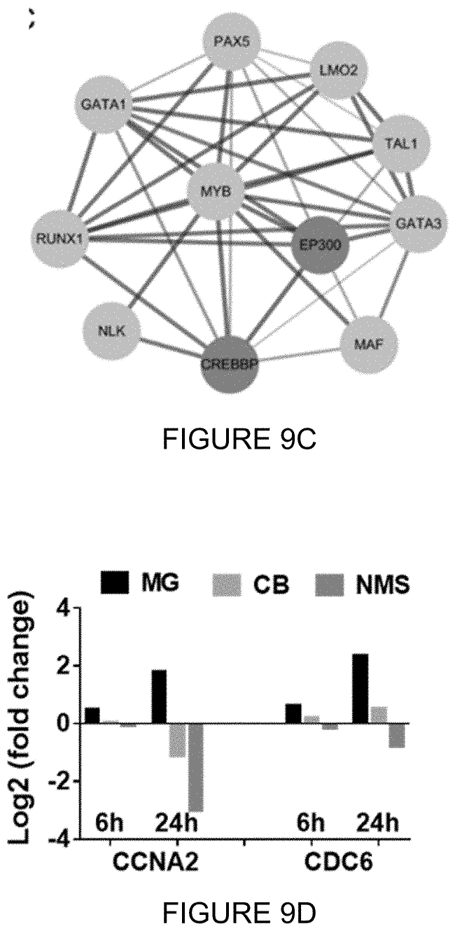

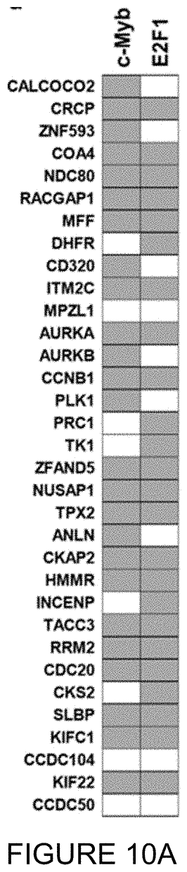

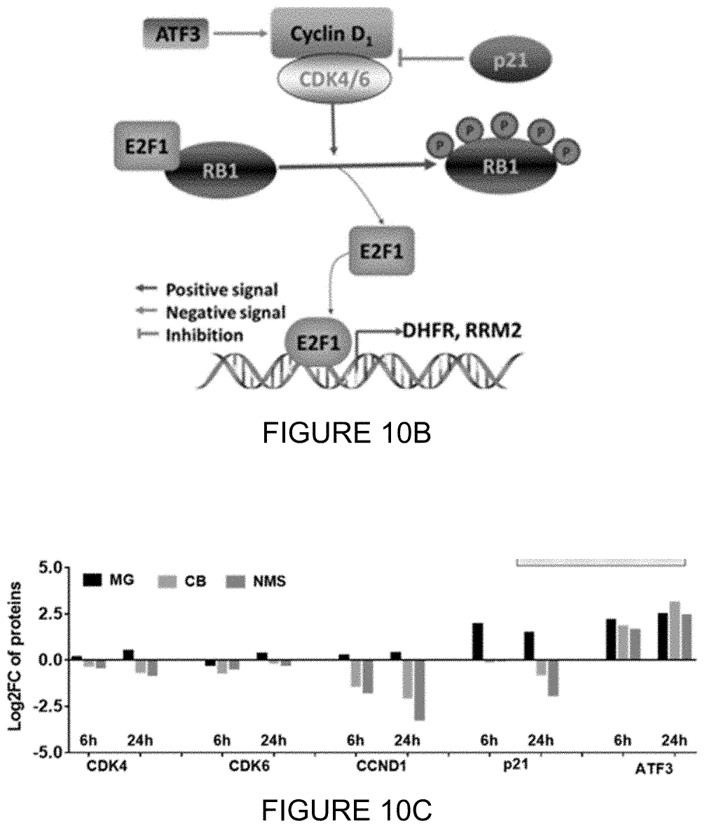

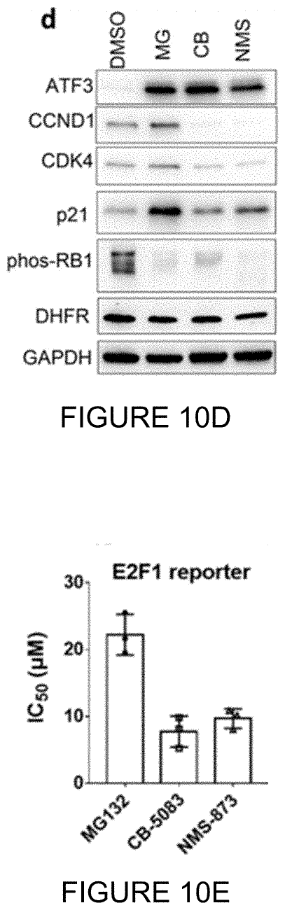

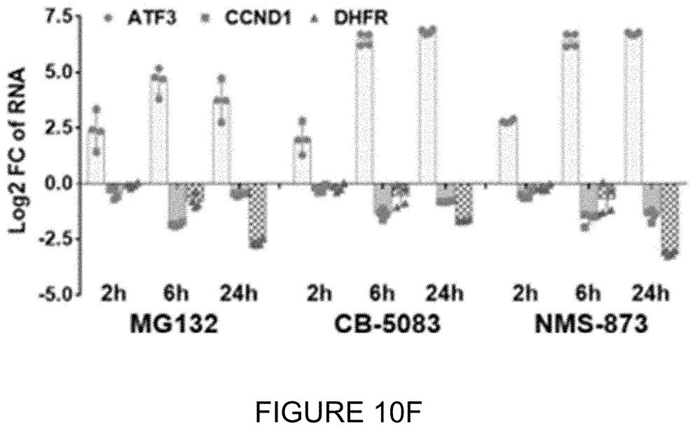

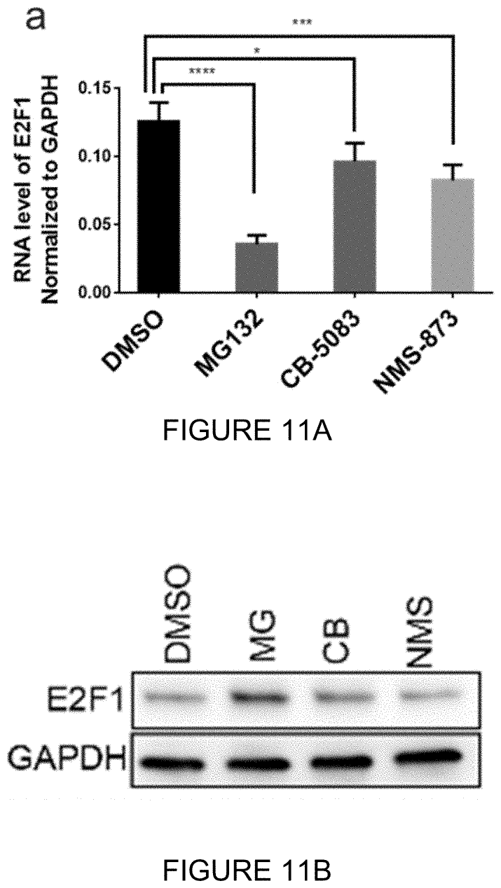

To further examine mechanisms specific to p97, rather than proteasome, inhibition analysis was focused on the proteins specifically downregulated by the former treatment. To investigate whether the protein translation was inhibited by p97 or proteasome inhibitor, the phosphorylation state of eukaryotic translation initiation factor 4E-binding protein 1 (4E-BP1) was examined. Phosphorylation of 4E-BP1 serves to increase total protein translation [40]. After 6 h of treatments, both MG132 and p97 inhibitors inhibit global protein translation, to a greater extent in MG132 treated cells (FIG. 9A). 20 of the 33 proteins identified are downregulated by NMS-873 at RNA levels in U2OS cells (FIG. 8B) in a previous dataset which sought to identify general p97 substrates [38]. The effect of p97 inhibition on specific protein levels arises from a change in transcript levels. To examine how transcript levels might be affected, potential transcription factor binding sites (TFBSs) in the promoters of these 33 genes in the TRANSFAC database were examined. This search revealed putative binding sites for two transcription factors, E2F1 and MYB. By examining protein-protein interaction networks related to E2F1 and MYB in the STRING database, 11 E2F1 interactors were found (FIG. 9B) and 11 MYB interactors (FIG. 9C). Two proteins, Cyclin A2 (CCNA2) and CDC6, are E2F1 interactors and are also significantly dysregulated in the TMT proteomic analysis following proteasome and p97 inhibition (FIG. 9D). In addition, some of the proteins downregulated by p97 inhibition (FIG. 10A), such as DHFR, TK1 and RRM2, have previously been experimentally validated as E2F1-specific target genes [41-43]. Therefore it was determined to further investigate the role of E2F1, rather than MYB, in cells responding to p97 and proteasome inhibition.

To determine if the decreased expression of E2F1 target genes was caused by the downregulation of E2F1 levels due to ER stress [44], qPCR and western blot were used to evaluate E2F1 levels. E2F1 RNA levels were strongly downregulated by MG132 and slightly downregulated by p97 inhibitors (FIGS. 11A-11B), consistent with the fact that ER stress induces the downregulation of E2F1. However, the protein level of E2F1 was not significantly affected by p97 inhibitors and was increased by MG132 (FIG. 11A). The transcriptional activity of E2F1 can also be repressed through the formation of a complex with the tumor suppressor protein retinoblastoma (RB1) (FIG. 10B) [45]. Hyperphosphorylation of RB1 by cyclin-D-cyclin-dependent kinase 4/6 complex (CCND1-CDK4/6) inactivates RB1 and induces the release of the E2F1 from the complex [46]. The newly released E2F1 leads to transcriptional activation of target genes [47]. From the proteomic analysis, CCND1 (cyclin D1) and CDK4/6 were all significantly downregulated by p97 inhibitors but upregulated by MG132 (FIG. 10C). This downregulation of CCND1 and CDK4 was further confirmed by western blot (FIG. 10D). By performing a published E2F1 reporter assay [48], it was found that both MG132 and p97 inhibitors reduce the transcriptional activity of E2F1, and the IC50 of p97 inhibitors is 2-fold lower than that of MG132 (FIG. 10E). Additionally, qPCR analysis indicates that mRNA levels of the E2F1 target gene DHFR are also downregulated by p97 inhibitors (FIG. 10F). This data indicates that the transcriptional activity of E2F1 is decreased by p97 inhibition and leading to downregulation of E2F1 target genes in some embodiments. Furthermore, the downregulation of CCND1 occurred after the upregulation of ATF3 (FIG. 10F). ATF3 is a stress-inducible gene, which binds to the cyclin D1 promoter and represses its transcription [49]. Therefore, the upregulation of ATF3 potentially contribute to the downregulation of CCND1.

It was hypothesized that CCND1 and CDK4/6 upregulation by MG132 promotes RB1 hyperphosphorylation. However, the western blots showed that MG132 treatment instead reduced phosphorylation of RB1 in addition to reducing the mRNA level of DHFR (FIGS. 10E and 10F), indicating that the transcriptional activity of E2F1 was also inhibited by MG132. To reveal why the upregulated CCND1-CDK4/6 complex does not promote phosphorylation of RB1 (FIGS. 10B and 10C), the CKI (cyclin-dependent kinase inhibitor) was focused on and it was found that p21 (CDKN1A) was significantly upregulated by MG132 but not by p97 inhibitors (FIGS. 10C and 10D). This indicates that in some embodiments, upregulation of p21 can block the increased activity of CCND1-CDK4/6 complex in MG132 treated cells and reduce RB1 phosphorylation.

Taken together, the results indicate that in some embodiments, HCT116 cells respond to proteasome and p97 inhibition by upregulating ATF3 mRNA and protein levels (FIGS. 10C, 10D and 10E) leading to a subsequent reduction in CCND1 mRNA (FIG. 10E). Unlike proteasome inhibition which increases cyclin D1 and CDK4/6, what distinguishes p97 inhibition is the reduction of all three of these oncoproteins at the protein level. The results indicate that both cyclin D1 and p21 play an important role in regulating the E2F1 pathway in cells treated with p97 inhibitors or MG132. Overall, both MG132 and p97 inhibitors lead to the reduction of unphosphorylated RB1 which, as a result, sequesters E2F1 and blocks its transcriptional activity.

### Example 5: p97 Inhibition Promotes the Downregulation of Cell Cycle Oncoproteins

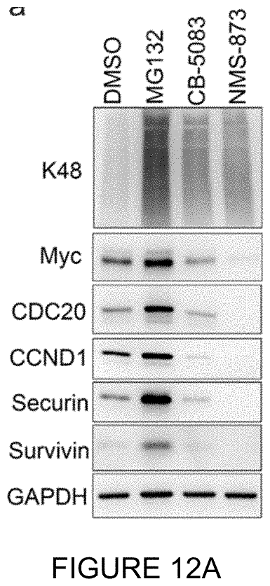

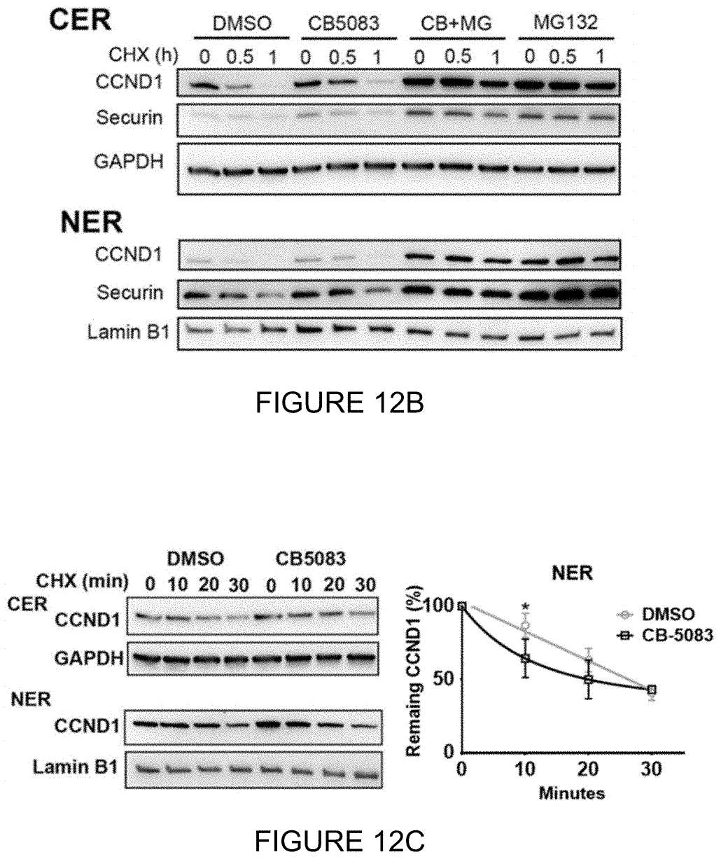

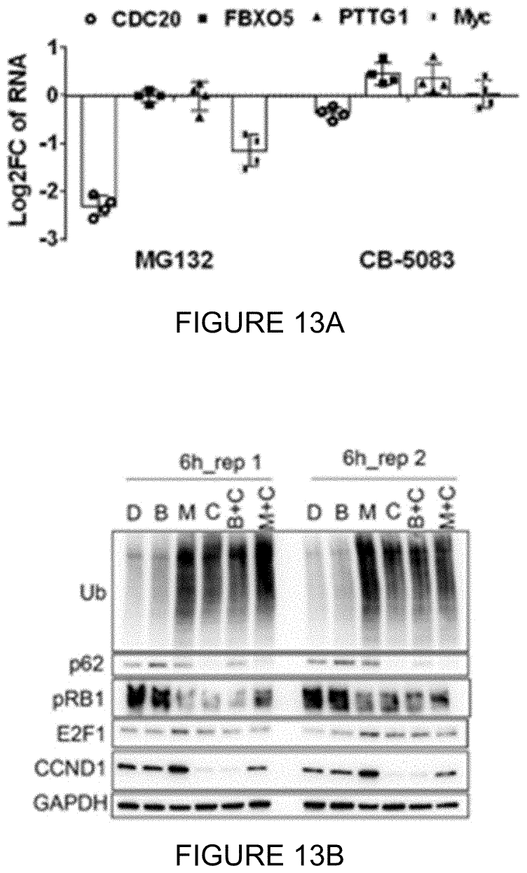

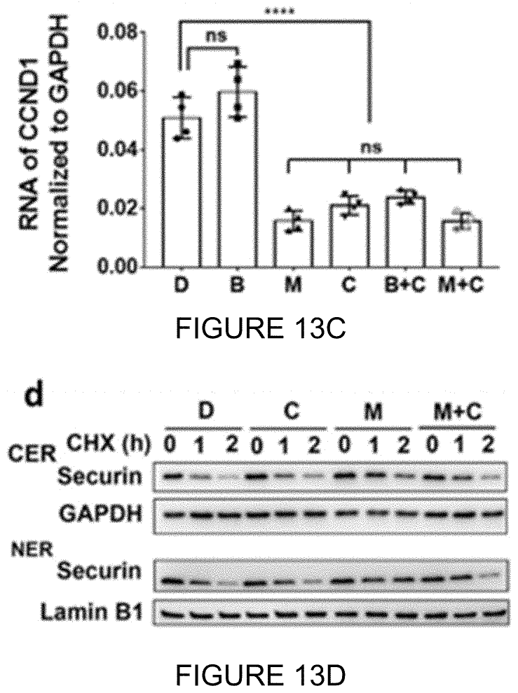

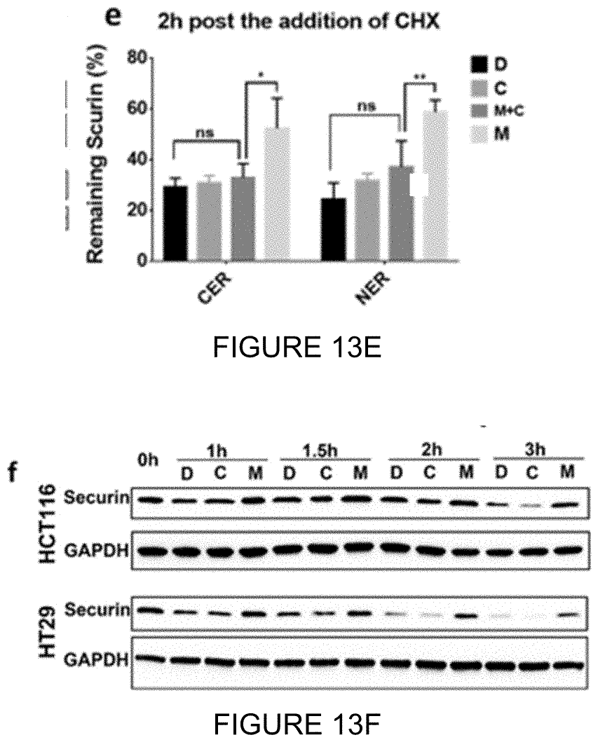

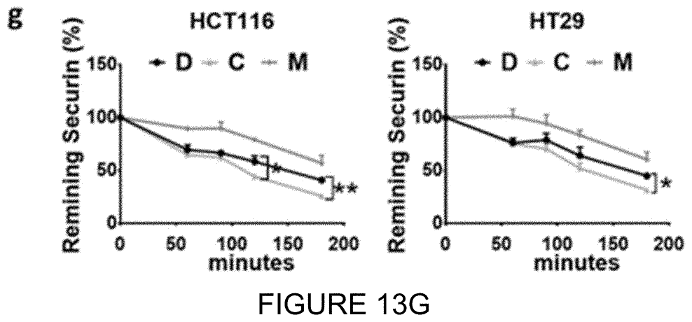

Functional enrichment analysis on proteins specifically downregulated by p97 inhibitors revealed that cell cycle factors are highly associated with p97 inhibition (FIG. 6E). To follow up on this effect, TMT experiments for cell cycle proteins that are significantly differentially expressed were examined. It was found that eleven cell cycle proteins—Securin (PTTG1), Cyclin D1 (CCND1), MYC, Survivin (BIRC5), Emil (FBXO5), CDC20, Bub1, CDC25B, ORC6, GMNN, RRM2—are specifically downregulated after 6 h and 24 h treatment with p97 inhibitors only (Table 1). To exclude cell-specific effects, the representative cell cycle proteins, Cyclin D1, Myc, Securin, Survivin and CDC20 were detected in HT29 cells treated with p97 inhibitors or MG132, and it was found all the five proteins displayed similar changes as observed in HCT116 cells (FIG. 12A-12C). To probe the effect at the transcriptional level, the RNA levels of Myc, Securin, Emil and CDC20 were determined after 6 h treatment (FIG. 13A) and it was found that only CDC20 was downregulated by CB-5083. The most affected two proteins, cyclin D1 and Securin, were focused on to investigate how protein levels are affected by both proteasome and p97 inhibition.

Since p97 is involved in both the proteasomal and autophagy degradation pathways, it was next tested which process is linked to the changes seen in cyclin D1 levels upon p97 inhibition. Cells were co-treated with CB-5083 and either the proteasome inhibitor MG132 or the autophagy inhibitor Bafilomycin A1 (Baf A1). The results showed that MG132 treatment rescued CB-5083 mediated cyclin D1 downregulation at the protein level but not at the mRNA level while Baf A1 had no effect on cyclin D1 levels (FIGS. 13B and 13C). In addition, there was a greater reduction in cyclin D1 protein in samples co-treated with CB-5083 and MG132 than those treated with MG132 treatment alone. This result indicates that in some embodiments, CB-5083 promotes cyclin D1 degradation in HCT116 cells. Cyclin D1 degradation in the cytosol and nucleus was further examined (FIG. 12B) and it was found that in both the cytosol and nucleus, cyclin D1 turnover was blocked by MG132. Due to the short half-life of cyclin D1 (less than 30 minutes), it was found that CB-5083 only slightly accelerated nuclear, but not cytosolic, cyclin D1 degradation in HCT116 cells (FIG. 13C).

Securin protein degradation from the cytosol and nucleus was also examined and it was found that it was blocked by MG132 (FIG. 13D, FIG. 12B). In addition, CB-5083 and MG132 co-treatment reduced the stabilization effect of MG132 on Securin (FIGS. 13D and 13E), indicating that, in some embodiments, the degradation of Securin does not depend on p97. When the cells were pretreated with MG132 for 1 h to buildup Securin, CB-5083 slightly accelerated the degradation of Securin in both HCT116 and HT29 cells (FIGS. 13F and 13G). It was confirmed that cyclin D1 and Securin are proteasome substrates and do not require p97 for degradation by proteasome. Overall, p97 inhibitors did not increase the protein level of these cell cycle oncoproteins, and instead downregulated protein levels. These results distinguish the role of p97 from that of the proteasome in regulating cell cycle proteins.

TABLE 1
Cell cycle proteins specifically downregulated by p97 inhibitors after 6 h and 24 h treatment ^{a}

| | | | Log₂ FC | | | | | | |
|---|---|---|---|---|---|---|---|---|---|
| | | Gene | 6 h | | | 24 h | | | |
| Protein | Description | name | MG | CB | NMS | MG | CB | NMS | cell cycle |
| O95997 | Securin | PTTG1 | 1.4 | −0.9 | −1.2 | −0.1 | −1.9 | −2.9 | M |
| P24385 | G1/S-specific cyclin- | CCND1 | 0.2 | −1.4 | −1.8 | 0.4 | −2.0 | −3.2 | G1/S |
| | D1 | | | | | | | | |
| P01106 | Myc proto-oncogene | MYC | 1.2 | −0.5 | −1.1 | 0.2 | −0.7 | −1.1 | G1/S |
| | protein | | | | | | | | |
| O15392 | Baculoviral IAP | BIRC5 | 0.6 | −0.5 | −0.5 | 1.0 | −0.8 | −2.0 | M/checkpoint |
| | repeat-containing | | | | | | | | |
| | protein 5 | | | | | | | | |
| Q9UKT4 | F-box only protein 5 | FBXO5 | 0.2 | −0.8 | −1.1 | 1.5 | −1.0 | −2.4 | M/G1 |
| Q12834 | Cell division | CDC20 | 0.8 | −0.4 | −0.6 | 1.1 | −1.5 | −2.8 | M/checkpoint |
| | cycle protein 20 | | | | | | | | |
| | homolog | | | | | | | | |
| O43683 | Mitotic checkpoint | BUB1 | 1.2 | −0.3 | −0.4 | 1.4 | −1.1 | −2.1 | M/checkpoint |
| | serine/threonine- | | | | | | | | |
| | protein kinase BUB1 | | | | | | | | |
| P30305 | M-phase inducer | CDC25B | 0.3 | −1.1 | −1.6 | −0.2 | −1.6 | −2.2 | S/G2/M |
| | phosphatase 2 | | | | | | | | |
| Q9Y5N6 | Origin recognition | ORC6 | 0.3 | −0.4 | −0.5 | 1.0 | −0.3 | −0.6 | G1/S/checkpoint |
| | complex subunit 6 | | | | | | | | |
| O75496 | Geminin | GMNN | 0.9 | −0.4 | −0.6 | 1.4 | −1.1 | −1.8 | G1/S |
| P31350 | Ribonucleoside- | RRM2 | 0.6 | −0.2 | −0.3 | 1.2 | −1.1 | −2.7 | G1 |
| | diphosphate reductase | | | | | | | | |
| | subunit M2 | | | | | | | | |

^{a} Data was collected from TMT labeling proteomics, Log₂FC indicates log₂ fold change (compound vs DMSO). 6 h and 24 h indicate the duration of treatment on the cells, MG (MG132), CB (CB-5083), NMS (NMS-873).

While previous studies have used proteomics to examine potential p97 partners and substrates in HEK293, and 293T cells, none of these have obtained temporal profiles and examined mechanisms that are potentially linked to the specific therapeutic effects of p97 inhibition [50-52]. Two studies used proteomic profiling in U2OS cell [38] and HEK293 cells [39]. One study performed proteomic profiling of NMS-873 after 6 h treatment in HCT116 cells [53]. These studies identified interesting p97 functions and substrates that are degraded in a p97-dependent manner. Here, however, a systematic analysis of p97 inhibition over time was conducted, using genetic knockdown and three small molecule inhibitors with different binding modes. Previous studies have also shown that p97/VCP is involved in multiple cellular processes [54] and that p97 inhibition is a potentially promising therapeutic strategy for treating cancers, neurodegenerative disease and virus infection [24]. However, most published studies on the cellular effects of p97 inhibition have focused on one or a few aspects of p97 functions, such as p97-adaptor binding [39, 53] and the degradation of ubiquitinated proteins through endoplasmic-reticulum-associated protein degradation (ERAD) [55]. Here a mass spectrometry-based quantitative proteomics approach was used to reveal a comprehensive picture of the proteomic and cellular pathways altered in HCT116 cells in response to both genetic and pharmacological p97 inhibition. The proteomic profiles of p97 inhibition to those of proteasome inhibition at several time points were then compared.

These examples demonstrate the overall influence of p97 inhibition on cellular functions (FIG. 2C and FIG. 2F), and show that p97 KD upregulates formation of proteasome as well as E2 ubiquitin-conjugating enzyme complexes to compensate for reduced proteolytic activity (FIG. 2D). In addition, the proteomic changes induced by p97 shRNA KD were compared to those induced by three pharmacological p97 inhibitors and inhibition by CB-5083 and NMS-873 was found to be more similar to p97 KD than UPCDC-30245. Specifically, UPR and factors associated with protein processing in the ER pathway were upregulated by both CB-5083 and NMS-873 as well as p97 KD (as previously reported), but not by UPCDC-30245 (FIG. 2G) [17, 20, 22, 53, 56-58]. This result is consistent with previous study that demonstrated by western blot analysis of two UPR markers (CHOP and ATF4) and two autophagy markers (p62 and LC3) [20].

The cell fate decisions under ER stress could be divided into two stages, the adaptive response phase (early stage) and apoptotic phase (late stage) [60, 61]. During the early stage, global protein translation is inhibited by PERK [62] and selective degradation of mRNA [63, 64] and autophagy is activated [65] to reduce the influx of proteins into the ER and re-establish homeostasis. Prolonged stress conditions and CHOP activation results in pro-death signaling overweighing pro-survival signaling, which leads to apoptosis [61, 66]. Temporal proteomics was used to identify representative time points for the different cell fate decisions induced by p97 inhibition. 6 h and 24 h provide representative time points that reveal early and late proteomic changes in response to p97 inhibition (FIG. 3A).

The proteomic profile of p97 inhibition was compared to the proteomic profile of proteasome inhibition. Proteasome inhibition and p97 inhibition had a similar effect on some protein clusters (FIG. 6C). However, p97 inhibitors activated the XBP1 pathway more potently than MG132 (FIG. 6G and FIG. 6H), consistent with what has been reported in models of multiple myeloma [37]. In addition, both TAX1BP1 and p62 were downregulated by p97 inhibitors but upregulated by MG132 after 6 h treatment, revealing differential effects of p97 and proteasome inhibition on autophagy (FIG. 3B). This supports the notion that upregulation of autophagy is a possible mechanism by which resistance to proteasome inhibition emerges. In addition, this reveals that p97 inhibition blocks two major proteasome degradation pathways [16]. TMBIM6 (Bax inhibitor 1) enhances autophagy via regulating lysosomal calcium and accelerates p62 degradation [67]. The rapid upregulation of TMBIM6 by p97 inhibitors, but not by MG132, may explain the downregulation of p62 at 6 h (FIG. 8A and FIG. 8D).

The most striking difference between p97 and proteasome inhibition is that many cell cycle proteins were specifically downregulated by p97 inhibition. Since the discovery of yeast p97 via isolation of yeast temperature-sensitive Cdc48 mutants [68], the role of p97 in regulating the cell cycle has focused on its ability to extract cell cycle proteins from complexes and deliver them to the proteasome for degradation. Based on this, p97 and the proteasome are determined herein to regulate the cell cycle as part of the same pathway. For example, they are both recruited to K11/K48 ubiquitinylated H2B, promoting its degradation and maintaining cell identify during cell division [69]. Studies have reported both p97 inhibition and MG132 treatment lead to HCT116 cells arresting in the G2-M phase [17, 70]. Two exemplary differences between the effect of p97 inhibition and proteasome inhibition on cell cycle regulation are described herein:

1. The mechanism underlying E2F1 inhibition by p97 inhibitors is different from MG132. p97 ATPase activity is required to maintain levels of the CCND1-CDK4/6 complex, whereas proteasome activity degrades p21 protein, and therefore promotes active CCND1-CDK4/6 complex. The overall effect is the same for both inhibitors, reduced Rb 1 phosphorylation and E2F1 sequestration and thus reduced mRNA levels of the E2F1 target genes.

2. That p97 inhibitors and proteasome inhibitors have important opposing effects on the protein levels of several cell cycle that are also oncoproteins (Table 1). Although both MG132 and p97 inhibitors were reported to affect the cell cycle (FIG. 7A), the proteomics data provided herein show that the molecular mechanism by which they affect cell cycle is different. Two proteins were selected, including cyclin D1 and Securin, and their half lives in the presence of p97 or proteasome inhibitors was determined. Although stabilization of cyclin D1 and Securin with MG132 was expected, as both are proteasome substrates, p97 inhibitors do not stabilize degradation of cyclin D1 and Securin. Congruently, Parisi et al. showed that p97 and yeast Cdc48 prevent degradation of ubiquitinated cyclin D1 and that the first generation p97 inhibitor, DBeQ, promotes cyclin D1 degradation [71].

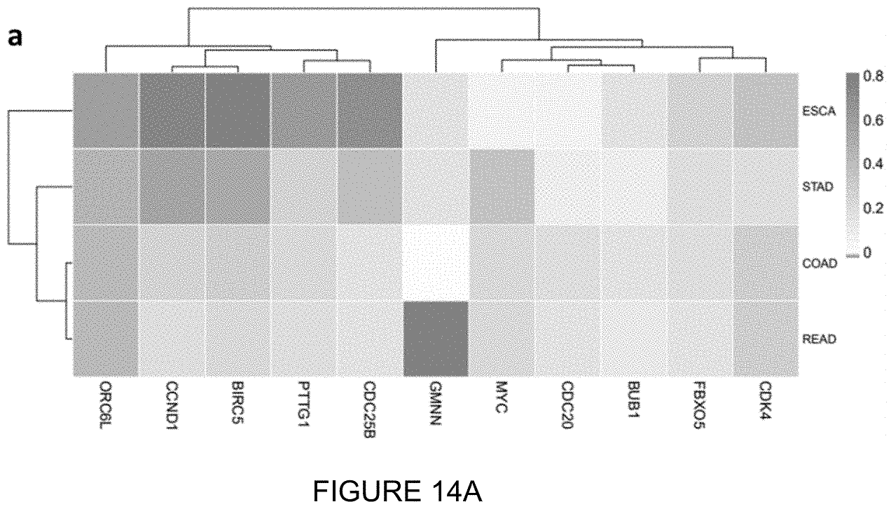

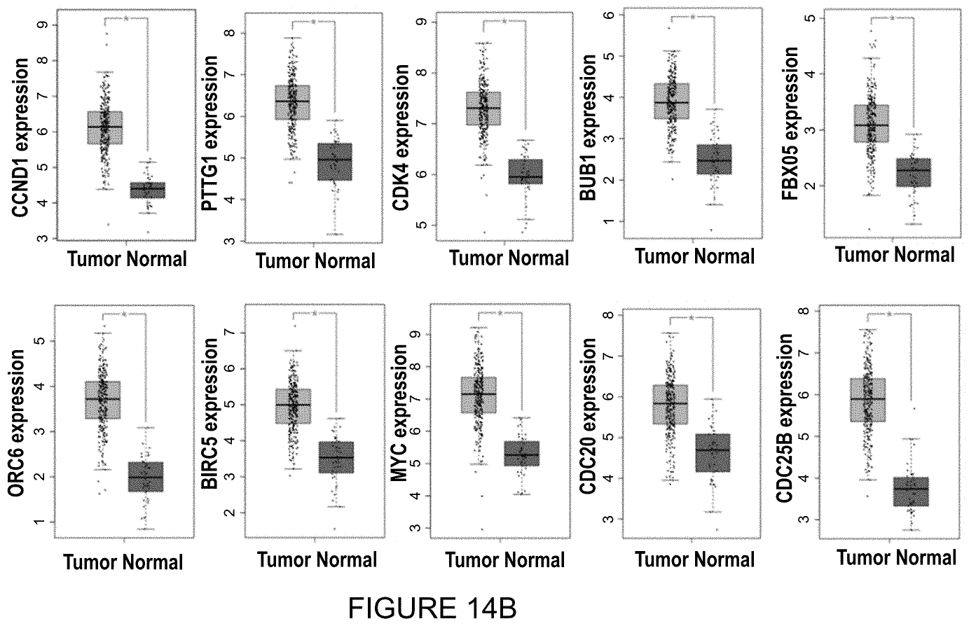

One of the major hallmarks of cancer is upregulation of cell cycle oncoproteins. The CCND-CDK4/6-INK4-Rb pathway is more frequently dysregulated in solid tumors and plays a central role in tumorigenesis and progression [72]. Of the eleven cell-cycle proteins specifically downregulated by p97, ten are significantly upregulated in TCGA-COAD patient tumors compared to normal matched tissue and most are upregulated in other GI cancers as well. (FIGS. 14A-14B). In addition, overexpression of some these proteins, such as Cyclin D1 is an unfavorable prognostic factor and is associated with tumor size and metastasis [73]. Mutations that directly perturb the degradation of Cyclin D1 and its nuclear export are also frequently observed in esophageal and uterine cancers [74]. Therefore, targeting the Cyclin D1-CDK4/6-INK4-Rb pathway represents a valid anticancer treatment in a broad spectrum of solid tumors and numerous inhibitors of CDK4/6 are currently under development and in clinical trials [73]. Inhibition of p97 impairs the Cyclin D1-CDK4/6-INK4-Rb pathway and the transcriptional activity of E2F1. That leads to the direct downregulation of cell cycle proteins and the deficit cell cycle. In some embodiments, the deficit cell cycle further amplifies the downregulation of cell cycle proteins. Taken together, the down regulation of cell cycle proteins explains why p97 inhibitors were effective in solid tumor models [22] while proteasome inhibitors, which stabilizes cell cycle oncoproteins, are largely ineffective.

A better understanding of the diversity and complexity of ubiquitin signaling in cell cycle regulation will shed new light on the precise control of cell cycle progression and guide anticancer drug development [75]. Embodiments of the methods described herein provide an unexpected and surprising value of chemical tools in combination with accurate temporal proteomic measurement. Embodiments of the methods described herein demonstrate p9′7's critical role in regulating many cell cycle oncoproteins, solidifying its potential as an excellent drug target in cancers that exhibit upregulated cell cycle oncoproteins.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

### REFERENCES

1. Cheng, J., et al., The emerging roles of protein homeostasis governing pathways in Alzheimer's disease. Aging Cell, 2018. 17(5): p. e12801.
2. Deshaies, R. J., Proteotoxic crisis, the ubiquitin-proteasome system, and cancer therapy. BMC Biol, 2014. 12: p. 94.
3. Ye, Y., et al., A Mighty “Protein Extractor” of the Cell: Structure and Function of the p97/CDC48 ATPase. Front Mol Biosci, 2017. 4: p. 39.
4. Ye, Y., et al., A membrane protein complex mediates retro-translocation from the ER lumen into the cytosol. Nature, 2004. 429(6994): p. 841-7.
5. Wu, X. and T. A. Rapoport, Mechanistic insights into ER-associated protein degradation. Curr Opin Cell Biol, 2018. 53: p. 22-28.
6. Wolf, D. H. and A. Stolz, The Cdc48 machine in endoplasmic reticulum associated protein degradation. Biochim Biophys Acta, 2012. 1823(1): p. 117-24.
7. van den Boom, J. and H. Meyer, VCP/p97-Mediated Unfolding as a Principle in Protein Homeostasis and Signaling. Mol Cell, 2018. 69(2): p. 182-194.
8. Ravanelli, S., F. den Brave, and T. Hoppe, Mitochondrial Quality Control Governed by Ubiquitin. Front Cell Dev Biol, 2020. 8: p. 270.
9. Meyer, H. H., Golgi reassembly after mitosis: the AAA family meets the ubiquitin family. Biochim Biophys Acta, 2005. 1744(2): p. 108-19.
10. Ramanathan, H. N. and Y. Ye, The p97 ATPase associates with EEA1 to regulate the size of early endosomes. Cell Res, 2012. 22(2): p. 346-59.
11. Kaneko, Y., et al., p97 and p47 function in membrane tethering in cooperation with FTCD during mitotic Golgi reassembly. EMBO J, 2021: p. e105853.
12. Meyer, H. H., Y. Wang, and G. Warren, Direct binding of ubiquitin conjugates by the mammalian p97 adaptor complexes, p47 and Ufd1-Npl4. EMBO J, 2002. 21(21): p. 5645-52.
13. Papadopoulos, C., B. Kravic, and H. Meyer, Repair or Lysophagy: Dealing with Damaged Lysosomes. J Mol Biol, 2020. 432(1): p. 231-239.
14. Papadopoulos, C., et al., VCP/p97 cooperates with YOD1, UBXD1 and PLAA to drive clearance of ruptured lysosomes by autophagy. EMBO J, 2017. 36(2): p. 135-150.
15. Arhzaouy, K., et al., VCP maintains lysosomal homeostasis and TFEB activity in differentiated skeletal muscle. Autophagy, 2019. 15(6): p. 1082-1099.
16. Chou, T. F., et al., Reversible inhibitor of p97, DBeQ, impairs both ubiquitin-dependent and autophagic protein clearance pathways. Proc Natl Acad Sci USA, 2011. 108(12): p. 4834-9.
17. Magnaghi, P., et al., Covalent and allosteric inhibitors of the ATPase VCP/p97 induce cancer cell death. Nat Chem Biol, 2013. 9(9): p. 548-56.
18. Alverez, C., et al., Structure Activity Study of Bioisosteric Trifluoromethyl and Pentafluorosulfanyl Indole Inhibitors of the AAA ATPase p97. ACS Med Chem Lett, 2015. 6(12): p. 1225-30.
19. LaPorte, M. G., et al., Optimization of Phenyl Indole Inhibitors of the AAA+ATPase p97. ACS Med Chem Lett, 2018. 9(11): p. 1075-1081.
20. Wang, F., et al., Allosteric p97 Inhibitors Can Overcome Resistance to ATP-Competitive p97 Inhibitors for Potential Anticancer Therapy. ChemMedChem, 2020. 15(8): p. 685-694.
21. Chou, T. F., et al., Structure-activity relationship study reveals ML240 and ML241 as potent and selective inhibitors of p97 ATPase. ChemMedChem, 2013. 8(2): p. 297-312.
22. Anderson, D. J., et al., Targeting the AAA ATPase p97 as an Approach to Treat Cancer through Disruption of Protein Homeostasis. Cancer Cell, 2015. 28(5): p. 653-665.
23. Zhou, H. J., et al., Discovery of a First-in-Class, Potent, Selective, and Orally Bioavailable Inhibitor of the p97 AAA ATPase (CB-5083). J Med Chem, 2015. 58(24): p. 9480-97.
24. Huryn, D. M., D. J. P. Kornfilt, and P. Wipf, p97: An Emerging Target for Cancer, Neurodegenerative Diseases, and Viral Infections. J Med Chem, 2020. 63(5): p. 1892-1907.
25. Yang, C. H., et al., Bortezomib (VELCADE) in metastatic breast cancer: pharmacodynamics, biological effects, and prediction of clinical benefits. Ann Oncol, 2006. 17(5): p. 813-7.
26. Engel, R. H., et al., A phase II study of single agent bortezomib in patients with metastatic breast cancer: a single institution experience. Cancer Invest, 2007. 25(8): p. 733-7.
27. Schmid, P., et al., A phase I/II study of bortezomib and capecitabine in patients with metastatic breast cancer previously treated with taxanes and/or anthracyclines. Ann Oncol, 2008. 19(5): p. 871-6.
28. Trinh, X. B., et al., A phase II study of the combination of endocrine treatment and bortezomib in patients with endocrine-resistant metastatic breast cancer. Oncol Rep, 2012. 27(3): p. 657-63.
29. Li, Z., et al., Systematic comparison of label-free, metabolic labeling, and isobaric chemical labeling for quantitative proteomics on LTQ Orbitrap Velos. J Proteome Res, 2012. 11(3): p. 1582-90.
30. Shrimal, S., et al., Asparagine-linked glycosylation is not directly coupled to protein translocation across the endoplasmic reticulum in Saccharomyces cerevisiae. Mol Biol Cell, 2019. 30(21): p. 2626-2638.
31. Dall'Olio, G. M., et al., The annotation of the asparagine N-linked glycosylation pathway in the Reactome database. Glycobiology, 2011. 21(11): p. 1395-400.
32. Steffen, J., et al., Proteasomal degradation is transcriptionally controlled by TCF11 via an ERAD-dependent feedback loop. Mol Cell, 2010. 40(1): p. 147-58.
33. Radhakrishnan, S. K., W. den Besten, and R. J. Deshaies, p97-dependent retrotranslocation and proteolytic processing govern formation of active Nrf1 upon proteasome inhibition. Elife, 2014. 3: p. e01856.
34. Akopian, D. and M. Rape, Conducting the finale of DNA replication. Genes Dev, 2017. 31(3): p. 226-227.
35. Li, Z. and X. Xu, Post-Translational Modifications of the Mini-Chromosome Maintenance Proteins in DNA Replication. Genes (Basel), 2019. 10(5): p. 331.
36. Schweppe, D. K., et al., Full-Featured, Real-Time Database Searching Platform Enables Fast and Accurate Multiplexed Quantitative Proteomics. J Proteome Res, 2020. 19(5): p. 2026-2034.
37. Le Moigne, R., et al., The p97 Inhibitor CB-5083 Is a Unique Disrupter of Protein Homeostasis in Models of Multiple Myeloma. Mol Cancer Ther, 2017. 16(11): p. 2375-2386.
38. Heidelberger, J. B., et al., Proteomic profiling of VCP substrates links VCP to K6-linked ubiquitylation and c-Myc function. EMBO Rep, 2018. 19(4): p. e44754.
39. Xue, L., et al., Valosin-containing protein (VCP)-Adaptor Interactions are Exceptionally Dynamic and Subject to Differential Modulation by a VCP Inhibitor. Mol Cell Proteomics, 2016. 15(9): p. 2970-86.
40. Walejko, J. M., et al., Branched-chain alpha-ketoacids are preferentially reaminated and activate protein synthesis in the heart. Nat Commun, 2021. 12(1): p. 1680.
41. Wells, J., et al., The identification of E2F1-specific target genes. Proc Natl Acad Sci USA, 2002. 99(6): p. 3890-5.
42. Chen, T., et al., The retinoblastoma protein selectively represses E2F1 targets via a TAAC DNA element during cellular senescence. J Biol Chem, 2012. 287(44): p. 37540-51.
43. Fang, Z., et al., E2F1 promote the aggressiveness of human colorectal cancer by activating the ribonucleotide reductase small subunit M2. Biochem Biophys Res Commun, 2015. 464(2): p. 407-15.
44. Pagliarini, V., et al., Downregulation of E2F1 during ER stress is required to induce apoptosis. J Cell Sci, 2015. 128(6): p. 1166-79.
45. McNair, C., et al., Differential impact of RB status on E2F1 reprogramming in human cancer. J Clin Invest, 2018. 128(1): p. 341-358.
46. Burkhart, D. L. and J. Sage, Cellular mechanisms of tumour suppression by the retinoblastoma gene. Nat Rev Cancer, 2008. 8(9): p. 671-82.
47. Lange, C. A. and D. Yee, Killing the second messenger: targeting loss of cell cycle control in endocrine-resistant breast cancer. Endocr Relat Cancer, 2011. 18(4): p. C19-24.
48. Neuman, E., et al., Transcription of the E2F-1 gene is rendered cell cycle dependent by E2F DNA-binding sites within its promoter. Mol Cell Biol, 1994. 14(10): p. 6607-15.
49. Lu, D., C. D. Wolfgang, and T. Hai, Activating transcription factor 3, a stress-inducible gene, suppresses Ras-stimulated tumorigenesis. J Biol Chem, 2006. 281(15): p. 10473-81.
50. Alexandru, G., et al., UBXD7 binds multiple ubiquitin ligases and implicates p97 in HIF1alpha turnover. Cell, 2008. 134(5): p. 804-16.
51. Raman, M., et al., Systematic proteomics of the VCP-UBXD adaptor network identifies a role for UBXN10 in regulating ciliogenesis. Nat Cell Biol, 2015. 17(10): p. 1356-69.
52. Rosenow, E. C., 3rd, Diffuse pulmonary infiltrates in the immunocompromised host. Clin Chest Med, 1990. 11(1): p. 55-64.
53. Her, N. G., et al., p97 Composition Changes Caused by Allosteric Inhibition Are Suppressed by an On-Target Mechanism that Increases the Enzyme's ATPase Activity. Cell Chem Biol, 2016. 23(4): p. 517-28.
54. Stach, L. and P. S. Freemont, The AAA+ATPase p97, a cellular multitool. Biochem J, 2017. 474(17): p. 2953-2976.
55. Braun, R. J. and H. Zischka, Mechanisms of Cdc48/VCP-mediated cell death: from yeast apoptosis to human disease. Biochim Biophys Acta, 2008. 1783(7): p. 1418-35.
56. Bastola, P., et al., VCP inhibitors induce endoplasmic reticulum stress, cause cell cycle arrest, trigger caspase-mediated cell death and synergistically kill ovarian cancer cells in combination with Salubrinal. Mol Oncol, 2016. 10(10): p. 1559-1574.
57. Zhao, Z., et al., CB-5083, an inhibitor of P97, suppresses osteosarcoma growth and stem cell properties by altering protein homeostasis. Am J Transl Res, 2020. 12(6): p. 2956-2967.
58. Garrison, P. and J. D. Bangs, p97 Inhibitor CB-5083 Blocks ERAD in Trypanosoma brucei. Mol Biochem Parasitol, 2020. 239: p. 111313.
59. Chou, T. F. and R. J. Deshaies, Development of p97 AAA ATPase inhibitors. Autophagy, 2011. 7(9): p. 1091-2.
60. Hetz, C., The unfolded protein response: controlling cell fate decisions under ER stress and beyond. Nat Rev Mol Cell Biol, 2012. 13(2): p. 89-102.
61. Corazzari, M., et al., Endoplasmic Reticulum Stress, Unfolded Protein Response, and Cancer Cell Fate. Front Oncol, 2017. 7: p. 78.
62. Harding, H. P., et al., Regulated Translation Initiation Controls Stress-Induced Gene Expression in Mammalian Cells. Molecular Cell, 2000. 6(5): p. 1099-1108.
63. Hollien, J., et al., Regulated Ire I-dependent decay of messenger RNAs in mammalian cells. J Cell Biol, 2009. 186(3): p. 323-31.
64. Hollien, J. and J. S. Weissman, Decay of endoplasmic reticulum-localized mRNAs during the unfolded protein response. Science, 2006. 313(5783): p. 104-7.
65. Kroemer, G., G. Marino, and B. Levine, Autophagy and the integrated stress response. Mol Cell, 2010. 40(2): p. 280-93.
66. Hetz, C. and F. R. Papa, The Unfolded Protein Response and Cell Fate Control. Mol Cell, 2018. 69(2): p. 169-181.
67. Kim, H. K., et al., TMBIM6 (transmembrane BAX inhibitor motif containing 6) enhances autophagy through regulation of lysosomal calcium. Autophagy, 2020: p. 1-18.
68. Moir, D., et al., Cold-sensitive cell-division-cycle mutants of yeast: isolation, properties, and pseudoreversion studies. Genetics, 1982. 100(4): p. 547-63.
69. Oh, E., et al., Gene expression and cell identity controlled by anaphase-promoting complex. Nature, 2020. 579(7797): p. 136-140.
70. Kim, O. H., et al., Influence of p53 and p2 IWaf1 expression on G2/M phase arrest of colorectal carcinoma HCT116 cells to proteasome inhibitors. International journal of oncology, 2004. 24(4): p. 935-941.
71. Parisi, E., et al., Cdc48/p97 segregase is modulated by cyclin-dependent kinase to determine cyclin fate during G1 progression. EMBO J, 2018. 37(16).
72. Schettini, F., et al., CDK 4/6 Inhibitors as Single Agent in Advanced Solid Tumors. Front Oncol, 2018. 8: p. 608.
73. Qie, S. and J. A. Diehl, Cyclin D1, cancer progression, and opportunities in cancer treatment. J Mol Med (Berl), 2016. 94(12): p. 1313-1326.
74. Benzeno, S., et al., Identification of mutations that disrupt phosphorylation-dependent nuclear export of cyclin D1. Oncogene, 2006. 25(47): p. 6291-6303.
75. Dang, F., L. Nie, and W. Wei, Ubiquitin signaling in cell cycle control and tumorigenesis. Cell Death Differ, 2021. 28(2): p. 427-438.

What is claimed is:

1. A method of measuring sensitivity of a subject to p97 inhibition, the method comprising:
identifying a subject having a cancer, or a symptom thereof; and
measuring an expression profile of oncoproteins in a biological sample obtained from the subject, wherein the measured expression profile of the oncoproteins differs from a normal expression profile from a healthy subject;
wherein the oncoproteins comprise cell cycle oncoproteins or oncoproteins of a Cyclin D1-CDK4 or 6-RB1-E2F1 pathway; and
wherein the oncoproteins of the Cyclin D1-CDK4 or 6-RB1-E2F1 pathway comprise Cyclin D1, CDK4, or ATF3.

2. The method of claim 1, wherein measuring the expression profile comprises measuring expression of an E2F1 target gene.

3. The method of claim 2, wherein the E2F1 target gene comprises RRM2, TK1, or DHFR.

4. The method of claim 1, wherein the cancer comprises a blood tumor, a solid tumor, a lymphoma, a myeloma, acute myeloid leukemia (AML), esophageal cancer, colon cancer, uterine cancer, or myelodysplastic syndrome (MDS).

5. The method of claim 1, wherein the cell cycle oncoproteins comprise Securin, MYC, Survivin, Emil, CDC20, Bub1, CDC25B, ORC6, GMNN, or RRM2.

6. The method of claim 1, further comprising administering an effective amount of an agent that inhibits p97 in the subject, wherein the cancer or a symptom thereof is reduced after the administering.

7. The method of claim 6, wherein the agent that inhibits p97 is an inhibitory nucleic acid molecule, a p97 binding antagonist, a genetic tool, or a small molecule inhibitor.

8. The method of claim 7, wherein the inhibitory nucleic acid molecule is an antisense nucleic acid.

9. The method of claim 7, wherein the inhibitory nucleic acid molecule is a siRNA.

10. The method of claim 7, wherein the inhibitory nucleic acid molecule is a shRNA.

11. The method of claim 7, wherein the inhibitory nucleic acid molecule corresponds to or is complementary to at least a fragment of nucleic acid encoding p97.

12. The method of claim 7, wherein the p97 binding antagonist inhibits the binding of p97 to its binding partners.

13. The method of claim 12, wherein the p97 binding antagonist is an antibody against p97 or a fragment of p97.

14. The method of claim 13, wherein the antibody is a monoclonal, polyclonal or an antibody fragment selected from the group consisting of Fab, Fab′-SH, Fv, scFv, and (Fab′)₂ fragments.

15. The method of claim 7, wherein the genetic tool is selected from the group consisting of a CRISPR/Cas9 system, a zinc finger nuclease system, a TALEN system, a homing endonucleases system, and a meganuclease system.

16. The method of claim 7, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.

17. A method of identifying a subject having a cancer with susceptibility to p97 inhibition, the method comprising:
detecting a level of a protein in a biological sample obtained from the subject, wherein the protein is Securin, CyclinD1, MYC, Survivin, Emil, CDC20, Bub1, CDC25B, ORC6, GMNN, RRM2, CDK4, ATF3, TK1, DHFR, or an ortholog thereof, or a combination thereof.

18. The method of claim 17, further comprising detecting a presence, a genetic change, or a level of the protein, wherein the protein is expressed differently or has a different genetic status than a sample obtained from a healthy subject.

19. A method of improving, ameliorating, or treating a cancer, the method comprising:
detecting the genetic status, level, or expression of a cell cycle oncoprotein or a Cyclin D1-CDK4 or 6-RB1-E2F1 pathway oncoprotein in a subject;
comparing the genetic status, level, or expression of the cell cycle oncoprotein or the Cyclin D1-CDK4 or 6-RB1-E2F1 pathway oncoprotein to the genetic status, level or expression of a cell cycle oncoprotein or a Cyclin D1-CDK4 or 6-RB1-E2F1 pathway oncoprotein in a healthy subject, wherein detection of an abnormal genetic status or a high level in the subject relative to the normal subject indicates the presence of a cancer in the subject; and
administering to the subject an effective amount of an agent that inhibits p97 in the subject, wherein the agent that inhibits p97 is an inhibitory nucleic acid molecule, a p97 binding antagonist, a genetic tool, or a small molecule inhibitor;
wherein the cancer or a symptom thereof is reduced after the administering, and
wherein the Cyclin D1-CDK4 or 6-RB1-E2F1 pathway oncoprotein is Cyclin D1, CDK4, TK1, DHFR, or ATF3.

20. The method of claim 19, wherein the cancer comprises a blood tumor, a solid tumor, a lymphoma, a myeloma, acute myeloid leukemia (AML), esophageal cancer, colon cancer, uterine cancer, or myelodysplastic syndrome (MDS).

21. The method of claim 19, wherein the cell cycle oncoprotein is Securin, MYC, Survivin, Emil, CDC20, Bub1, CDC25B, ORC6, GMNN, or RRM2.

22. The method of claim 19, wherein the inhibitory nucleic acid molecule is an antisense nucleic acid.

23. The method of claim 19, wherein the inhibitory nucleic acid molecule is a siRNA.

24. The method of claim 19, wherein the inhibitory nucleic acid molecule is a shRNA.

25. The method of claim 19, wherein the inhibitory nucleic acid molecule corresponds to or is complementary to at least a fragment of nucleic acid encoding p97.

26. The method of claim 19, wherein the p97 binding antagonist inhibits the binding of p97 to its binding partners.

27. The method of claim 26, wherein the p97 binding antagonist is an antibody against p97 or a fragment of p97.

28. The method of claim 27, wherein the antibody is a monoclonal, polyclonal or an antibody fragment selected from the group consisting of Fab, Fab′-SH, Fv, scFv, and (Fab′)₂ fragments.

29. The method of claim 19, wherein the genetic tool is selected from the group consisting of a CRISPR/Cas9 system, a zinc finger nuclease system, a TALEN system, a homing endonucleases system, and a meganuclease system.

30. The method of claim 19, wherein the small molecule inhibitor is CB-5083, NMS-873, NMS-859, DBeQ, MSC1094308, ML240, p97-IN-1, VCP/p97 inhibitor-1, ML241 hydrochloride, or UPCDC-30245.
